(19) United States
(12) Patent Application Publication
(10) Pub. No.: US 2015/0315645 A1
(43) Pub. Date: Nov. 5, 2015

(54) METHODS OF IDENTIFYING BIOMARKERS ASSOCIATED WITH OR CAUSATIVE OF THE PROGRESSION OF DISEASE

(71) Applicants: Douglas E. GAASTERLAND, Colonial Beach, VA (US); The Regents of the University of California, Oakland, CA (US)

(72) Inventors: Theresa GAASTERLAND, Del Mar, CA (US); Douglas E. GAASTERLAND, Colonial Beach, VA (US)

(73) Assignee: The Regents of the University of California, Oakland, CA (US)

(21) Appl. No.: 14/701,965

(22) Filed: May 1, 2015

Related U.S. Application Data
(60) Provisional application No. 61/988,202, filed on May 3, 2014.

(51) Int. Cl. C12Q 1/68

(52) U.S. Cl. CPC C12Q 1/6883; C12Q 2600/178; C12Q 2600/118; C12Q 2600/156; C12Q 2600/158

(57) ABSTRACT

Provided are methods of identifying biomarkers that cause or promote progression of disease. The successful application of the methods is demonstrated by the identification of biomarkers associated with and/or causative of the onset and/or progression and/or severity and/or recurrence of glaucoma and POAG. Many of these biomarkers were not previously associated with glaucoma or POAG. Predictive methods are also described, as well as applications in prognosis, diagnosis, and therapy. Testing for onset, progression, severity, and/or recurrence can be carried out. A key advantage in at least some embodiments is that a patient can receive earlier treatment for the disease such as POAG by use of the methods, screenings, and predictions described herein. Another key advantage in at least some embodiments is that a patient can receive more personalized or particular treatment for the disease such as POAG by use of the methods, screenings, and predictions described herein.

### CROSS-REFERENCE TO RELATED APPLICATIONS

This application claims the benefit under 35 U.S.C. §119(e) of U.S. Provisional Application No. 61/988,202 filed on May 3, 2014, which is hereby incorporated herein by reference in its entirety for all purposes.

### STATEMENT OF GOVERNMENTAL SUPPORT

This invention was made with government support under Grant Nos. EY020678 and EY022306, awarded by the National Eye Institute, National Institutes of Health. The government has certain rights in the invention.

### FIELD

Provided are methods of identifying genes that cause or promote progression of disease.

### BACKGROUND

Many systematic, chronic types of diseases exist for which better diagnoses and treatments are needed, including the disease of glaucoma in its various forms. In glaucoma, progressive optic nerve degeneration often causes progressive, irreversible visual impairment, and potential blindness. Glaucoma is one of the most prevalent causes of blindness in the United States. Types of glaucoma can be grouped as open-angle, angle closure, and secondary. It is estimated that in the United States in 2010, of those over age 40, open-angle glaucoma affected nearly 2.8 million people, and worldwide caused bilateral blindness in more than 4.4 million people [1]. Primary open-angle glaucoma (POAG) is the more frequent form of the disease in the United States, affecting nearly equal numbers of men and women [2]. Treatment to lower the intraocular pressure (IOP) inhibits progression of vision loss from glaucoma; yet it is not always totally successful, and it seldom reverses established damage [3,4]. Because treatment inhibits progression of visual function damage, early detection is important.

People with a first-degree relative with POAG have double, or greater, risk of developing the disease [5,6]. A small number of identified genes clearly underlie a limited number of glaucoma cases, including some with POAG. Some genes have been noted as involved in open angle glaucoma or neurodegeneration similar to that found in POAG through gene expression studies, model systems, linkage, and genome wide association studies (GWAS). Identification of causative glaucoma-associated genes is key to risk prediction, early detection, and eventual curative intervention. A major risk factor for visual system damage in POAG is elevated IOP arising from abnormal fluid dynamics in the eye, yet glaucomatous optic nerve degeneration occurs in the presence of normal IOP in about half of cases [7]. Of Caucasian POAG patients enrolled in the meta-analysis of the combined Genetic Etiologies of Primary-open Angle Glaucoma (GLAUGEN) and National Eye Institute Glaucoma Human Genetics Collaboration (NEIGHBOR) GWAS, 1669 cases had IOP ≧22 mmHg before treatment, and 720 had IOP <22 mmHg [8]. Genetic observations in these patients hint at the genetic complexity of POAG. Tissues that participate in aqueous dynamics, and thus IOP, are in the front of the eye while the retina and optic nerve, where vision damage occurs, are in the back of the eye. Both are involved in high pressure POAG or high pressure glaucoma (HPG). Thus, it makes sense to search broadly in the genome and across tissue systems for genetic explanations.

Association and linkage-based glaucoma genetics studies have identified loci contributing to susceptibility to glaucoma or to phenotypic features associated with risk of glaucoma, for example, large optic discs [9]. Genes including myocilin, CYP1B1, and optineurin lead to early onset, juvenile, or congenital glaucoma and some cases of adult-onset POAG. Susceptibility alleles in the LOXL1 gene confer risk of exfoliation open-angle glaucoma, where disease is secondary [10]. The NEIGHBOR GWAS found two loci strongly associated with optic nerve degeneration in POAG, CDKN2B-AS1 and SIX1/SIX6 [8]. Other GWAS have reported an association of the CDKN2B-AS1, CAV1/CAV2, TMCO1, and GAS7 loci with POAG [11,12]. Taken individually, these genes explain a limited portion of cases of POAG.

Additional references which discuss the genetics of glaucoma and POAG include: (1) Nowak et al., Biomed. Research Int'l, 2015, ID258281 [13], (2) Nowak et al., Arch Med. Sci. 6, Dec. 2014, [14] (3) US Patent Publication 2009/0035279, (4) US Patent Publication 2007/0172919, and (5) US Patent Publication 2004/0132795.

### SUMMARY

Briefly, a study of genetics is described and claimed herein wherein, in a preferred embodiment, a genome-wide, targeted sequencing of exons and flanking regions was carried out based on blood-derived DNA from patients with HPG. Briefly, a new method of constraint-based filtering and analysis based on technical and clinical criteria has been developed and applied. Briefly, a search—using the single nucleotide polymorphisms (SNPs) found within and near transcribed exons—is described and claimed for potentially causative genes in patients, including patients with genetically complex, chronic diseaseses such as eye disease, such as glaucoma. In a preferred embodiment, through genomic DNA sequencing and computational search, briefly, genome variations with markedly higher occurrence in HPG patients have been identified in comparison with general populations. Of the approximately 25,000 genes encoded in the human genome, briefly, this study in its preferred embodiment has identified about 140 genes containing about 160 variants overrepresented in HPG. Unexpectedly, in the preferred embodiment, most of these genes and their variants have not been previously connected with glaucoma.

In one aspect, provided are methods of identifying genes whose alleles are associative with or causative of the progression of a disease, comprising:

a) sequencing or reviewing multiple exomes from patients who have been diagnosed with the disease and one or more exomes from one or more individuals known not to have the disease, wherein the one or more exomes from one or more individuals known not to have the disease comprise one or more reference exomes;

b) selecting exomes sequenced and read with a fidelity of 4 or fewer mismatches per 100 bases, e.g., fewer than 3 or 2 mismatches per 100 bases;

c) selecting for genes having one or more site variants in the exomes from patients who have been diagnosed with the disease with one or more properties, e.g., 1, 2, 3, 4, 5, 6, 7, 8, 9, 10, 11, 12, 13, 14, 15, 16, 17, or 18 properties, selected from:
i) site variant is found in one or more patients;
ii) site variant is observed in a general population dataset;
iii) site variant is found in three or more patients;
iv) one or more reference exomes have the major allele;
v) site variant is the minor allele in reference exomes;
vi) site variant has only one alternate allele;
vii) site is within genome region with balanced G+C and A+T content;

viii) site is located outside low complexity genome regions;
ix) site is located in genome region with no paralog within 95% identity; and
x) site variant is located on chromosomes 1-22 or site variant is located on chromosome X or Y only if disease incidence is gender-biased;
xi) site was measured in 25 or more patients;
xii) site variant frequency in patients differs from general populations by more than expected measurement error, e.g., 0.05 (on a frequency scale from 0.00-1.00);
xiii) site variant frequency in patients exceeds general populations, e.g., by more than 0.10;
xiv) site variant is within a gene or regulatory regions influencing its expression as RNA or protein;
xv) site variant is within or near a gene expressed in tissues relevant to disease;
xvi) odds ratio 95% confidence interval lower bound calculated for the site from patient and reference general population frequencies is above 1.00;
xvii) frequency of site variant in patients is above a line fitted to filtered sites represented as datapoints where X is reference general population frequency and Y is patient frequency, e.g., fit with least squares linear regression; and
xviii) a p-value calculated with a 2×2 statistical test, e.g., Fisher's Exact Test, from numbers of alternate and reference alleles observed for the site in patients and in general population remains significant after correction for multiple testing.

In varying embodiments, the methods comprise selecting for genes having one or more site variants in the exomes from patients who have been diagnosed with the disease is carried out with nine or more properties, or twelve or more properties, or fifteen or more properties, or all eighteen of the properties identified above (i) to (xviii).

In a further aspect, provided are methods of identifying genes whose alleles are associative with or causative of the onset and/or progression and/or severity and/or recurrence of a disease, comprising:

a) sequencing or reviewing multiple exomes from patients who have been diagnosed with the disease and one or more exomes from one or more individuals known not to have the disease, wherein the one or more exomes from one or more individuals known not to have the disease comprise one or more reference exomes;

b) selecting exomes sequenced and read with a fidelity of 4 or fewer mismatches per 100 bases;

c) selecting for genes having one or more site variants in the exomes from patients who have been diagnosed with the disease, wherein the genes have one or more properties, e.g., 1, 2, 3, 4, 5, 6, 7, 8, 9, or 10 properties, selected from:
i) site variant is found in one or more patients;
ii) site variant is observed in a general population dataset;
iii) site variant is found in three or more patients;
iv) one or more reference exomes have the major allele;
v) site variant is the minor allele in reference exomes;
vi) site variant has only one alternate allele;
vii) site is within genome region with balanced G+C and A+T content;
viii) site is located outside low complexity genome regions;
ix) site is located in genome region with no paralog within 95% identity; and
x) site variant is located on chromosomes 1-22 or site variant is located on chromosome X or Y only if disease incidence is gender-biased.

d) selecting for genes having one or more site variants in the exomes from patients who have been diagnosed with the disease, wherein the genes have one or more properties, e.g., 1, 2, 3, 4, 5, 6, 7, or 8 properties, selected from:
i) site was measured in 25 or more patients;
ii) site variant frequency in patients differs from general populations by more than expected measurement error, e.g., 0.05 (on a frequency scale from 0.00-1.00);
iii) site variant frequency in patients exceeds general populations, e.g., by more than 0.10;
iv) site variant is within a gene or regulatory regions influencing its expression as RNA or protein;
v) site variant is within or near a gene expressed in tissues relevant to disease;
vi) odds ratio 95% confidence interval lower bound calculated for the site from patient and reference general population frequencies is above 1.00;
vii) frequency of site variant in patients is above a line fitted to filtered sites represented as datapoints where X is reference general population frequency and Y is patient frequency, e.g., fit with least squares linear regression; and
viii) a p-value calculated with a 2×2 statistical test, e.g., Fisher's Exact Test, from numbers of alternate and reference alleles observed for the site in patients and in general population remains significant after correction for multiple testing.

In varying embodiments of the method of identification, the disease is, for example, a systematic, chronic disease, such as, for example, a neurodegenerative disease, a cancer, a cardiovascular disease, an ocular disease, an immune disease, an autoimmune disease, an endocrinologic disease (e.g., diabetes), or an inflammatory disease (including chronic inflammatory). In some embodiments, the disease is a neurodegenerative disease. In some embodiments, the disease is an ocular disease. In some embodiments, the disease is primary open angle glaucoma (POAG). In some embodiments, the patients are symptomatic for the disease. In some embodiments, the method is computer implemented. In some embodiments, the site variants are selected from single nucleotide polymorphisms (SNPs), insertions, deletions and rearrangements. In some embodiments, the methods further comprise determining the expression levels of the genes from patient exomes and reference exomes. In some embodiments, the methods further comprise determining the expression levels of the microRNA from patient exomes and reference exomes. In some embodiments, the sequencing step comprises employing a next-generation sequencing (NGS) technique or method. In some embodiments, the methods further comprise selecting exomes sequenced and read with a fidelity of 4, 3, 2, 1 or fewer (e.g., no) mismatches per 100 bases. In some embodiments, the general population exome dataset is selected from or derived from one or more of 1000 Genomes (1000genomes.org), the Exome Sequencing Project (evs.gs.washington.edu/EVS/) datasets, UK10K (uk10k.org/), UCSC Genome Bioinformatics Site (genome.ucsc.edu/), other available public datasets, and proprietary datasets made available for comparison. In some embodiments, the methods further comprise weighting said selected genes according to predictive power rankings of the collection of signature biomarkers.

In a further aspect, provided are methods for predicting onset and/or progression and/or severity and/or recurrence of primary open angle glaucoma (POAG) in a subject, the method comprising:

(a) receiving allelic information and/or expression levels of a collection of signature biomarkers from a biological sample taken from said subject suspected of suffering POAG, wherein said collection of signature biomarkers comprises one or more genes and/or microRNAs, e,g., 2, 3, 4, 5, 6, 7, 8, 9, 10, 15, 20, 25, 30, 40, 45, 50, 55, 60, 65, 70, 75, 80, 85, 90, or more or all, selected from the group consisting of: AATF, ABI1, ABI3BP, ACTN2, ADAMTS15, ADCY2, AHNAK2, ANGEL2, ANKRD36, ANKRD36B, ANO5, AP1M1, ARHGAP30, ASTN1, ATP6V1E2, BAI3, CACNA1E, CACNA1I, CALM1, CCDC66, CD163, CDH13, CDH4, CDK17, CELF5, CHD8, CLCA4, CLEC7A, CLSTN2, CNNM2, CNOT6, COL23A1, COL4A2, CRTAC1, CTU2, CYBA, DCBLD2, DHCR7, DNAJB11, DPF3, DRD2, EBF2, ENO3, EPT1, ERI2, FDX1L, FJ22184, FOXD4, FOXRED2, FRYL, GAS7, GNG7, GOLGA3, GRIA1, GRID1, GRM4, HERC2, HLA-A, HLA-DRB1, IFI6, IMMT, INPP5D, ITGB4, KIAA0930, LACTB2, LCP2, LEMD3, LILRB2, LILRB3, LIN7A, LOC642846, LOC643387, LOC728537, LPHN3, LRP3, LRP4, LRRC37A, MAML3, MATR3, MCCC1, MCF2L, MEGF11, MGC21881, MINK1, MRPL23, MUC4, MYH9, MYO1E, N6AMT1, NBPF16, NOMO2, NUCKS1, PALM2, PCK1, PCM1, PDE4DIP, PML, POTEC, PPFIA2, PRKAG2, PRKCH, PRKD1, PRUNE2, R3HDM1, RABGAP1, RAD51B, RBFOX1, RIN3, SARDH, SCAF8, SEC14L1, SEL1L3, SEMA5A, SEMA5B, SIRT1, SLC30A8, SNTB1, SPN, SPRY1, SRRM2, TMPRSS13, TNRC18, TOR1A, TRIM58, TSPAN11, TXNRD1, UNC5B, USP20, USP6, VAC14, VARS2, VCAN, WASH1, XRCC5, ZDHHC7, ZMYND11, ZNF155, ZNF573, ZNF594, ZNF83, hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, hsa-miR-99b-5p, hsa-miR-1246, hsa-miR-1248, hsa-miR-130a, hsa-miR-130a-3p, hsa-miR-145, hsa-miR-145-3p, hsa-miR-148a, hsa-miR-148a-3p, hsa-miR-214, hsa-miR-214-3p, hsa-miR-216a, hsa-miR-224, hsa-miR-224-5p, hsa-miR-27a-5p, hsa-miR-31, hsa-miR-31-5p, hsa-miR-4448, hsa-miR-449a, hsa-miR-452, hsa-miR-452-5p, hsa-miR-455, hsa-miR-455-5p, hsa-miR-483, hsa-miR-483-3p, hsa-miR-483-5p, hsa-miR-549, hsa-miR-5584, hsa-miR-5584-5p, hsa-miR-574, hsa-miR-574-5p, hsa-miR-675, hsa-miR-675-3p, hsa-miR-767, hsa-miR-767-5p, hsa-miR-9, hsa-miR-9-3p, msa-miR-27a, hsa-let-7a, hsa-let-7a-2, hsa-let-7a-2-3p, and hsa-let-7c;

(b) applying the allelic information and/or expression levels to a predictive model relating allelic information and/or expression levels of said collection of signature biomarkers with onset of POAG; and (c) evaluating an output of said predictive model to predict onset of POAG in said individual; and/or

(c) applying the allelic information and/or expression levels to a predictive model relating allelic information and/or expression levels of said collection of signature biomarkers with progression of POAG; and (e) evaluating an output of said predictive model to predict progression of POAG in said individual; and/or

(d) applying the allelic information and/or expression levels to a predictive model relating allelic information and/or expression levels of said collection of signature biomarkers with severity of POAG; and (g) evaluating an output of said predictive model to predict severity of POAG in said individual; and/or

(e) applying the allelic information and/or expression levels to a predictive model relating allelic information and/or expression levels of said collection of signature biomarkers with recurrence of POAG; and (i) evaluating an output of said predictive model to predict recurrence of POAG in said individual. The relevant sequence identifications for these biomarkers, genes, and microRNAs are incorporated herein by reference.

In some embodiments of the methods of predicting, said collection of signature biomarkers comprises one or more genes selected from the biomarkers listed in Tables 4, 5 and/or 6. In varying embodiments, collection of signature biomarkers comprises one or more genes selected from the group consisting of: AATF, ABI1, ABI3BP, ACTN2, ADAMTS15, ADCY2, AHNAK2, ANGEL2, ANKRD36, ANKRD36B, ANO5, AP1M1, ARHGAP30, ASTN1, ATP6V1E2, BAI3, CACNA1E, CACNA1I, CALM1, CCDC66, CD163, CDH13, CDH4, CDK17, CELF5, CHD8, CLCA4, CLEC7A, CLSTN2, CNNM2, CNOT6, COL23A1, COL4A2, CRTAC1, CTU2, CYBA, DCBLD2, DHCR7, DNAJB11, DPF3, DRD2, EBF2, ENO3, EPT1, ERI2, FDX1L, FLJ22184, FOXD4, FOXRED2, FRYL, GAS7, GNG7, GOLGA3, GRIA1, GRID1, GRM4, HERC2, HLA-A, HLA-DRB1, IFI6, IMMT, INPP5D, ITGB4, KIAA0930, LACTB2, LCP2, LEMD3, LILRB2, LILRB3, LIN7A, LOC642846, LOC643387, LOC728537, LPHN3, LRP3, LRP4, LRRC37A, MAML3, MATR3, MCCC1, MCF2L, MEGF11, MGC21881, MINK1, MRPL23, MUC4, MYH9, MYO1E, N6AMT1, NBPF16, NOMO2, NUCKS1, PALM2, PCK1, PCM1, PDE4DIP, PML, POTEC, PPFIA2, PRKAG2, PRKCH, PRKD1, PRUNE2, R3HDM1, RABGAP1, RAD51B, RBFOX1, RIN3, SARDH, SCAF8, SEC14L1, SEL1L3, SEMA5A, SEMA5B, SIRT1, SLC30A8, SNTB1, SPN, SPRY1, SRRM2, TMPRSS13, TNRC18, TOR1A, TRIM58, TSPAN11, TXNRD1, UNC5B, USP20, USP6, VAC14, VARS2, VCAN, WASH1, XRCC5, ZDHHC7, ZMYND11, ZNF155, ZNF573, ZNF594, and ZNF83, wherein the position and allele of the genetic variation associated with and/or causative of POAG is as provided in Table 4. In varying embodiments, overexpression of one or more microRNAs selected from hsa-miR-1246, hsa-miR-1248, hsa-miR-130a, hsa-miR-130a-3p, hsa-miR-145, hsa-miR-145-3p, hsa-miR-148a, hsa-miR-148a-3p, hsa-miR-214, hsa-miR-214-3p, hsa-miR-216a, hsa-miR-224, hsa-miR-224-5p, hsa-miR-27a-5p, hsa-miR-31, hsa-miR-31-5p, hsa-miR-4448, hsa-miR-449a, hsa-miR-452, hsa-miR-452-5p, hsa-miR-455, hsa-miR-455-5p, hsa-miR-483, hsa-miR-483-3p, hsa-miR-483-5p, hsa-miR-549, hsa-miR-5584, hsa-miR-5584-5p, hsa-miR-574, hsa-miR-574-5p, hsa-miR-675, hsa-miR-675-3p, hsa-miR-767, hsa-miR-767-5p, hsa-miR-9, hsa-miR-9-3p, msa-miR-27a, hsa-let-7a, hsa-let-7a-2, hsa-let-7a-2-3p, and hsa-let-7c in the biological sample from the subject in comparison to a control sample from an individual known not to have POAG predicts negative outcome or onset and/or progression and/or severity and/or recurrence of POAG. In varying embodiments, the methods comprise further administering to the subject an inhibitory nucleic acid that reduces or inhibits the expression of one or more microRNAs selected from hsa-miR-1246, hsa-miR-1248, hsa-miR-130a, hsa-miR-130a-3p, hsa-miR-145, hsa-miR-145-3p, hsa-miR-148a, hsa-miR-148a-3p, hsa-miR-214, hsa-miR-214-3p, hsa-miR-216a, hsa-miR-224, hsa-miR-224-5p, hsa-miR-27a-5p, hsa-miR-31, hsa-miR-31-5p, hsa-miR-4448, hsa-miR-449a, hsa-miR-452, hsa-miR-452-5p, hsa-miR-455, hsa-miR-455-5p, hsa-miR-483, hsa-miR-483-3p, hsa-miR-483-5p, hsa-miR-549, hsa-miR-5584, hsa-miR-5584-5p, hsa-miR-574, hsa-miR-574-5p, hsa-miR-675, hsa-miR-675-3p, hsa-miR-767, hsa-miR-767-5p, hsa-miR-9, hsa-miR-9-3p, msa-miR-27a, hsa-let-7a, hsa-let-7a-2, hsa-let-7a-2-3p, and hsa-let-7c. In varying embodiments, the methods further comprise administering to the subject one or more microRNAs or one or more mimics of microRNAs selected from hsa-miR-1246, hsa-miR-1248, hsa-miR-130a, hsa-miR-130a-3p, hsa-miR-145, hsa-miR-145-3p, hsa-miR-148a, hsa-miR-148a-3p, hsa-miR-214, hsa-miR-214-3p, hsa-miR-216a, hsa-miR-224, hsa-miR-224-5p, hsa-miR-27a-5p, hsa-miR-31, hsa-miR-31-5p, hsa-miR-4448, hsa-miR-449a, hsa-miR-452, hsa-miR-452-5p, hsa-miR-455, hsa-miR-455-5p, hsa-miR-483, hsa-miR-483-3p, hsa-miR-483-5p, hsa-miR-549, hsa-miR-5584, hsa-miR-5584-5p, hsa-miR-574, hsa-miR-574-5p, hsa-miR-675, hsa-miR-675-3p, hsa-miR-767, hsa-miR-767-5p, hsa-miR-9, hsa-miR-9-3p, msa-miR-27a, hsa-let-7a, hsa-let-7a-2, hsa-let-7a-2-3p, and hsa-let-7c. In varying embodiments, underexpression or nonexpression of one or more microRNAs selected from hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, and hsa-miR-99b-5p in the biological sample from the subject in comparison to a control sample from an individual known not to have POAG predicts a negative outcome or onset and/or progression and/or severity and/or recurrence of POAG. In varying embodiments, the methods comprise further administering to the subject an inhibitory nucleic acid that reduces or inhibits the expression of one or more microRNAs selected from hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, and hsa-miR-99b-5p. In varying embodiments, the methods further comprise administering to the subject one or more microRNAs or one or more mimics of microRNAs selected from hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, and hsa-miR-99b-5p. In some embodiments, the individual is symptomatic for POAG. In some embodiments, the individual has a family history of POAG. In some embodiments, said output of the predictive model predicts a likelihood of recurrence of POAG in the individual after said individual has undergone treatment for POAG. In some embodiments, the methods further comprise providing a report having a prediction of clinical recurrence of POAG of said individual. In some embodiments, the methods further comprise combining the allelic information and/or gene expression levels of said signature biomarkers with one or more other biomarkers to predict onset and/or progression and/or severity and/or recurrence of POAG in said individual. In some embodiments, the expression levels of a collection of signature biomarkers comprise gene expression levels are measured at multiple times. In varying embodiments, the methods further comprise using the dynamics of the gene expression levels measured at multiple times to predict onset and/or progression and/or severity and/or recurrence of disease (e.g., HPG/POAG) in said subject. In varying embodiments, the methods further comprise evaluating the output of the predictive model to determine whether or not the individual falls in a high risk group. In varying embodiments, the methods further comprise developing said predictive model using stability selection or logistic regression. In varying embodiments, the methods further comprise developing said predictive model using stability selection. In varying embodiments, the methods further comprise developing said predictive model using logistic regression. In some embodiments, applying said allelic information and/or expression levels of the collection of signature biomarkers to said predictive model comprises weighting said expression levels according to stability rankings or predictive power rankings of the collection of signature biomarkers. In some embodiments, applying said allelic information and/or expression levels of the collection of signature biomarkers to said predictive model comprises weighting said expression levels according to stability rankings of the collection of signature biomarkers. In some embodiments, applying said allelic information and/or expression levels of the collection of signature biomarkers to said predictive model comprises weighting said expression levels according to predictive power rankings of the collection of signature biomarkers.

One embodiment is a method of identifying genes whose alleles are associative with or causative of the progression of a disease, comprising:

a) sequencing or reviewing multiple exomes from patients who have been diagnosed with the disease and one or more exomes from one or more individuals known not to have the disease, wherein the one or more exomes from one or more individuals known not to have the disease comprise one or more reference exomes;

b) selecting exomes sequenced and read with a fidelity of 4 or fewer mismatches per 100 bases;

c) selecting for genes having one or more site variants in the exomes from patients who have been diagnosed with the disease with one or more properties selected from:
i) site variant is present in 25 or more patients;
ii) site variant has only one alternate allele;
iii) the one or more reference exomes have the major allele;
iv) site variant is within a gene or regulatory regions influencing its expression as RNA or protein;
v) site variant is located on chromosomes 1-22 or site variant is located on chromosome X or Y only if disease incidence is gender-biased;
vi) site variant has a frequency of ≦0.95 in patients;
vii) site variant is within general population exome dataset;
viii) site variant has approximately the same frequency within the general population as the frequency of the disease within the general population; and
ix) site variant occurs in patients with a frequency greater than in the general population.

Another embodiment is a method of identifying genes whose alleles are associative with or causative of the progression of a disease, comprising:

a) sequencing or reviewing multiple exomes from patients who have been diagnosed with the disease and one or more exomes from one or more individuals known not to have the disease, wherein the one or more exomes from one or more individuals known not to have the disease comprise one or more reference exomes;

b) selecting exomes sequenced and read with a fidelity of 4 or fewer mismatches per 100 bases;

c) selecting for genes having one or more site variants in the exomes from patients who have been diagnosed with the disease with one or more properties selected from:
i) site variant is present in two or more patients;
ii) site variant has only one alternate allele;
iii) the one or more reference exomes have the major allele; and
iv) site variant is within a gene or regulatory regions influencing its expression as RNA or protein;

d) selecting for genes having one or more site variants in the exomes from patients who have been diagnosed with the disease, wherein the genes have one or more properties selected from:
i) site variant is present in 25 or more patients;
ii) site variant is located on chromosomes 1-22 or site variant is located on chromosome X or Y only if disease incidence is gender-biased;
iii) site variant has a frequency of ≦0.95 in patients;
iv) site variant is within general population exome dataset;
v) site variant has approximately the same frequency within the general population as the frequency of the disease within the general population; and
vi) site variant occurs in patients with a frequency greater than in the general population.

Still further, another embodiment is a method for predicting progression of primary open angle glaucoma (POAG) in a subject, the method comprising:

(a) receiving allelic information and/or expression levels of a collection of signature biomarkers from a biological sample taken from said subject suspected of suffering POAG, wherein said collection of signature biomarkers comprises one or more genes and/or microRNA selected from the group consisting of: ABI1, ABI3BP, AKT1, ANKRD36B, CADM2, CCDC33, CELA3A, CHMP7, CHRNA7, CLCNKB, CNNM2, CNTN2, COL4A2, CSMD2, CSPG4, DPF3, ENO3, EPHA10, FANCM, FAT3, FBN3, FDX1L, GAK, GAS7, GINS2, GLB1L3, GLIS1, GOLGA3, GOLGA6B, GTF2I, GYPE, HLA-DQB1, HLA-DRB1, IL1B, KCNQ1, KCNQ3, KLF12, KLRC4, LGALS9C, LILRB2, LILRB3, LOXL2, MMD, MRPL23, MUC4, NBPF3, NLRP9, NOMO2, NPIPL2, NSUN4, NUP153, OR2L3, PAK7, PALM2, PDLIM4, PLAC4, PLXNA2, POTEM, PPP1R14C, PRAMEF2, PRB4, PRICKLE4, PRKAG2, PTPRN2, RANGAP1, RBM23, RGPD1, RYR2, SEL1L3, SEPT9, SLC2A3, SLC35E2, SLC6A18, SLC6A3, SPN, SRCIN1, SULT1A2, SYN3, SYT3, TMEM120A, TMEM191B, TMPRSS13, USP20, USP41, WASH1, ZNF276, ZNF492, ZNF512B, ZNF594, ZNF83, hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, hsa-miR-99b-5p, hsa-miR-1246, hsa-miR-1248, hsa-miR-130a, hsa-miR-130a-3p, hsa-miR-145, hsa-miR-145-3p, hsa-miR-148a, hsa-miR-148a-3p, hsa-miR-214, hsa-miR-214-3p, hsa-miR-216a, hsa-miR-224, hsa-miR-224-5p, hsa-miR-27a-5p, hsa-miR-31, hsa-miR-31-5p, hsa-miR-4448, hsa-miR-449a, hsa-miR-452, hsa-miR-452-5p, hsa-miR-455, hsa-miR-455-5p, hsa-miR-483, hsa-miR-483-3p, hsa-miR-483-5p, hsa-miR-549, hsa-miR-5584, hsa-miR-5584-5p, hsa-miR-574, hsa-miR-574-5p, hsa-miR-675, hsa-miR-675-3p, hsa-miR-767, hsa-miR-767-5p, hsa-miR-9, hsa-miR-9-3p, msa-miR-27a, hsa-let-7a, hsa-let-7a-2, hsa-let-7a-2-3p, and hsa-let-7c;

(b) applying the allelic information and/or expression levels to a predictive model relating allelic information and/or expression levels of said collection of signature biomarkers with progression of POAG; and

(c) evaluating an output of said predictive model to predict progression of POAG in said individual.

Also provided herein are methods of diagnosis, prognosis, and/or therapy for the diseases described herein, including glaucoma and POAG, and also methods and kits for determining the presence or absence of the disease, such as glaucoma or POAG, or of an increased risk of the disease, such as glaucoma or POAG in an individual. Methods for diagnosis, prognosis, and/or therapy for the diseases described herein, including glaucoma and POAG, are generally known in the art and can be combined with the methods of gene and biomarker identification described herein. For example, a patient can be tested for having or not having the identified genetic marker as described herein. One or more samples can be taken from the patient, and the samples analyzed. If the patient has the marker, additional diagnosis, prognosis, and therapy can be carried out with the patient. For example, one can analyze for onset, progression, severity, and/or recurrence of the disease. Methods known in the art can be used. See, for example, US Patent Publication 2004/0132795 for methods of screening and treating individuals with glaucoma or the propensity to develop glaucoma, and this reference is incorporated herein by reference in its entirety. Diseases other than glaucoma and POAG can be included in these methods. See, for example, US Patent Publication 2011/0177509 (which is incorporated herein by reference in its entirety) for risk factors and a therapeutic target for neurodegenerative disorders, as well as methods for identification of a subject at risk for a neurodegenerative disorder; see also U.S. Pat. No. 7,794,933 for neurological disorders including depression (and which is incorporated herein by reference in its entirety).

Kits designed and configured for practicing methods are also provided herein as known in the art of diagnostic and testing kits and devices. The use of kits is generally known in the art. See, for example, US Patent Publication 2011/0177509, which is incorporated herein by reference in its entirety. Kits can include, for example, appropriate genetic materials, indicators, instructions, and/or packaging.

Hence, also provided herein is a method of identifying a patient or subject using the methods described herein which can include kits. One or more genetic tests can be used to identify the patient or subject. The patient or subject can then be given a prognosis and/or treatment.

### DEFINITIONS

The term “exome” refers to the part of the genome formed by exons, the sequences which when transcribed remain within the mature RNA after introns are removed by RNA splicing. It differs from a transcriptome in that it consists of all DNA that is transcribed into mature RNA in cells of any type. For the purposes of the present application, the exome includes coding exons, non-coding exons, 5′ untranslated regions (UTR), 3′ UTR, flanking introns, microRNA, and proximal promoters.

The term “threshold level” refers to a representative or predetermined expression level of a gene or microRNA. The threshold level can represent expression detected in a sample from a normal control, i.e., from non-diseased tissue or non-diseased subject. In varying embodiments, the normal control is of the same tissue type of the biological sample subject to testing. The threshold level can be determined from an individual or from a population of individuals. The expression levels of a gene or microRNA from a diseased tissue or subject may be above (increased) or below (decreased) in comparison to a control level.

The terms “increased expression level” or “overexpression” interchangeably refer to a predetermined threshold level or a level of expression from a normal or non-diseased control. An increased expression level is determined when the level of expression in the test biological sample is at least about 10%, 25%, 50%, 75%, 100% (i.e., 1-fold), 2-fold, 3-fold, 4-fold or greater, in comparison to the predetermined threshold level of expression or the level of expression from a normal or non-diseased control tissue. In determining an increased level of expression, usually the same tissue types are compared.

The terms “decreased expression level” or “underexpression” interchangeably refer to a predetermined threshold level or a level of expression from a normal or non-diseased control. A decreased expression level is determined when the level of expression in the test biological sample is at least about 10%, 25%, 50%, 75%, 100% (i.e., 1-fold), 2-fold, 3-fold, 4-fold or less or lower, in comparison to the predetermined threshold level of expression or the level of expression from a normal or non-diseased control tissue. In determining an decreased level of expression, usually the same tissue types are compared.

The term “individual,” “patient,”, “subject” interchangeably refer to a mammal, for example, a human, a non-human primate, a domesticated mammal (e.g., a canine or a feline), an agricultural mammal (e.g., equine, bovine, ovine, porcine), or a laboratory mammal (e.g., rattus, murine, lagomorpha, hamster).

As used herein the term “comprising” means that the named elements are included, but other elements (e.g., unnamed signature genes) may be added and still represent a composition or method within the scope of the claim. The transitional phrase “consisting essentially of” means that the associated composition or method encompasses additional elements, including, for example, additional signature genes, that do not affect the basic and novel characteristics of the disclosure.

As used herein, the term “signature gene” refers to a gene whose expression is correlated, either positively or negatively, with disease extent or outcome or with another predictor of disease extent or outcome. In some embodiments, a gene expression score (GEX) can be statistically derived from the expression levels of a set of signature genes and used to diagnose a condition or to predict clinical course. In some embodiments, the expression levels of the signature genes may be used to predict onset and/or progression and/or severity and/or recurrence of disease (e.g., POAG or HPG) without relying on a GEX. A “signature nucleic acid” is a nucleic acid comprising or corresponding to, in case of cDNA, the complete or partial sequence of a RNA transcript encoded by a signature gene, or the complement of such complete or partial sequence. A signature protein is encoded by or corresponding to a signature gene of the disclosure.

The term “prediction” is used herein to refer to the prediction of disease onset and/or progression and/or severity and/or recurrence in a patient. The patient may be symptomatic or asymptomatic. The patient may have undergone or currently be undergoing a therapeutic regime. The predictive methods of the present disclosure can be used clinically to make treatment decisions by choosing the most appropriate treatment modalities for any particular patient. The predictive methods of the present disclosure also can provide valuable tools in predicting if a patient is likely to respond favorably to a treatment regimen, such as surgical intervention and/or pharmacological intervention.

The term “plurality” refers to more than one element. For example, the term is used herein in reference to a number of nucleic acid molecules or sequence tags that are sufficient to identify significant differences in copy number variations in test samples and qualified samples using the methods disclosed herein. In some embodiments, at least about 3×10⁶ sequence tags of between about 20 and 40 bp are obtained for each test sample. In some embodiments, each test sample provides data for at least about 5×10⁶, 8×10⁶, 10×10⁶, 15×10⁶, 20×10⁶, 30×10⁶, 40×10⁶, or 50×10⁶ sequence tags, each sequence tag comprising between about 20 and 40 bp.

The terms “polynucleotide,” “nucleic acid” and “nucleic acid molecules” are used interchangeably and refer to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The nucleotides include sequences of any form of nucleic acid, including, but not limited to RNA and DNA molecules. The term “polynucleotide” includes, without limitation, single- and double-stranded polynucleotide.

The terms “microRNA mimic” and “mimics of microRNA” are well known in the art. See e.g., Wang, Z., 2009, Chapter on “miRNA Mimic Technology,” pages 93-100, MicroRNA Interference Technologies, Springer-Verlag. Herein, it can refer to synthetic sequences that are nearly identical or identical to microRNAs found in cells. They can be, for example, sometimes modified chemically in some way for stability (e.g., to make it through the liver) or with a nucleotide or two changed for delivery or manufacturing purposes. Herein, microRNAs or short synthetic RNAs nearly identical to the microRNAs can be used, e.g., 90% identical or closer, possibly with chemical modifications to the nucleotides. Double stranded miRNA mimics can be used.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “read” refers to a sequence read from a portion of a nucleic acid sample. Typically, though not necessarily, a read represents a short sequence of contiguous base pairs in the sample. The read may be represented symbolically by the base pair sequence (in ATCG) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene.

As used herein, the terms “aligned,” “alignment,” or “aligning” refer to the process of comparing a read or tag to a reference sequence and thereby determining whether the reference sequence contains the read sequence. If the reference sequence contains the read, the read may be mapped to the reference sequence or, in certain embodiments, to a particular location in the reference sequence. In some cases, alignment simply tells whether or not a read is a member of a particular reference sequence (i.e., whether the read is present or absent in the reference sequence). For example, the alignment of a read to the reference sequence for human chromosome 13 will tell whether the read is present in the reference sequence for chromosome 13. A tool that provides this information may be called a set membership tester. In some cases, an alignment additionally indicates a location in the reference sequence where the read or tag maps to. For example, if the reference sequence is the whole human genome sequence, an alignment may indicate that a read is present on chromosome 13, and may further indicate that the read is on a particular strand and/or site of chromosome 13.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. One example of an algorithm from aligning sequences is the Efficient Local Alignment of Nucleotide Data (ELAND) computer program distributed as part of the Illumina Genomics Analysis pipeline. Alternatively, a Bloom filter or similar set membership tester may be employed to align reads to reference genomes. Alternatively, an indexing algorithm such as that implemented in versions of the BowTie computer program may be employed to align reads to reference genomes. The matching of a sequence read in aligning can be a 100% sequence match or less than 100% (non-perfect match).

The term “mapping” used herein refers to specifically assigning a sequence read to a larger sequence, e.g., a reference genome, by alignment.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. A “genome” refers to the complete genetic information of a mammal expressed in nucleic acid sequences.

In various embodiments, the reference sequence is significantly larger than the reads that are aligned to it. For example, it may be at least about 100 times larger, or at least about 1000 times larger, or at least about 10,000 times larger, or at least about 10⁵ times larger, or at least about 10⁶ times larger, or at least about 10⁷ times larger.

The term “based on” when used in the context of obtaining a specific quantitative value, herein refers to using another quantity as input to calculate the specific quantitative value as an output.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

The term “condition” herein refers to “medical condition” as a broad term that includes all diseases and disorders, but can include [injuries] and normal health situations, such as pregnancy, that might affect a person's health, benefit from medical assistance, or have implications for medical treatments.

The term “sensitivity” as used herein is equal to the number of true positives divided by the sum of true positives and false negatives.

The term “specificity” as used herein is equal to the number of true negatives divided by the sum of true negatives and false positives.

### BRIEF DESCRIPTION OF THE DRAWINGS

FIG. 1 illustrates strategies for high fidelity identification of SNPs, insertions/deletions (indels), and genome rearrangements associated with disease causation and/or progression. Upper left (SNPS @ 3×): Large rectangles represent ranges of genome nucleotides to which sequence reads, represented by smaller lines, were mapped. To identify SNPs, reads with 0 to 3 mismatches per 100 bases are aligned to the reference genome and their bases are compared. Mismatches between reference nucleotides and read nucleotides, represented by dark dots on the reads, designate a variant site. Generally, 3+ sequence reads are needed to determine whether a site has a variant. Upper right (indel): Reads that span a small insertion or deletion in a patient genome are aligned to a reference genome with gaps in the read or reference. Lower (split pair): Paired reads may fail to align nearby each other in the reference genome because of a rearrangement in a patient genome. Their alignment to different genome regions, on the same or different chromosome, depicted by left and right large rectangles indicates a rearrangement.

FIG. 2 illustrates genes with their strength of expression in human eye tissues. Left: Dark to light color represents high to low overall expression in eye tissues for a non-exhaustive list of genes detected as expressed in eye tissues by RNA sequencing; genes were selected to range from high to low expression. Three genes previously associated with glaucoma are noted, GAS7, HLA-DRB1, and COL4A2. Right: Also depicted is expression of GAS7 in 6 eye tissues, including trabecular meshwork (TM), cilliary bodies (CB), choroid (CH), optic disk (OD), optic nerve (ON) and retina (RT) with stronger expression in TM, OD, ON, and RT compared to CB and CH. Blue lines (top) denote gene exons. Black vertical lines denote RNA sequence reads.

FIG. 3 illustrates expression of four genes in 6 eye tissues, for each gene including trabecular meshwork (TM), ciliary body (CB), choroid (CH), optic disk (OD), optic nerve (ON) and retina (RT). Each gene has distinct tissue-specific expression.

FIG. 4 provides a scatterplot of filtered variant sites represented as datapoints with X=frequency of variant in general populations and Y=frequency of variant in HPG patients.

FIG. 5 illustrates microRNA overexpressed in diseased optic nerve (i.e., optic nerve from patients having primary open angle glaucoma). Overexpressed microRNAs include hsa-miR-483-5p, hsa-miR-483-3p, hsa-miR-214-3p, hsa-miR-452-5p, hsa-miR-4448, hsa-miR-224-5p, hsa-miR-1246, hsa-miR-130a-3p, hsa-miR-9-3p, hsa-miR-767-5p, and hsa-miR-449a.

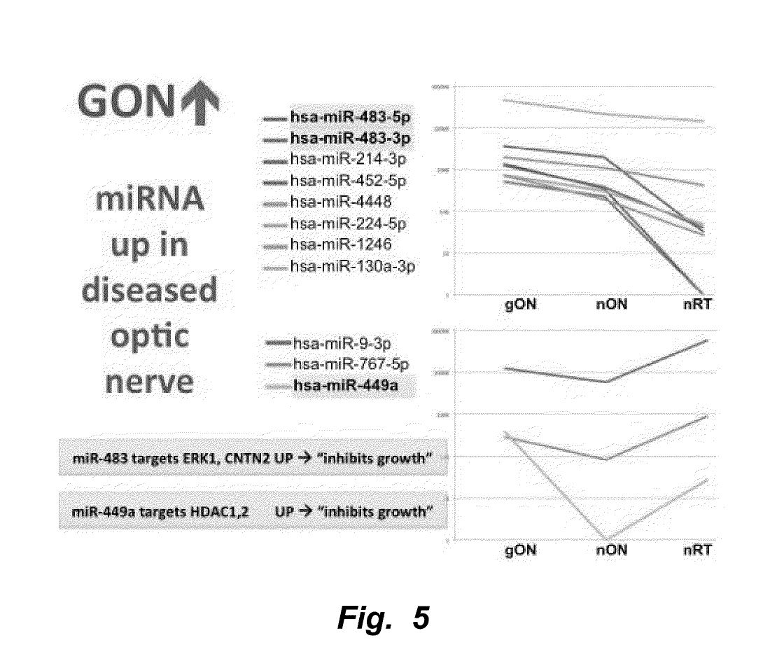

FIG. 6 illustrates microRNA (miRNA) underexpressed in diseased optic nerve (i.e., optic nerve from patients having primary open angle glaucoma). Underexpressed microRNAs include hsa-miR-34b-3p, hsa-miR-3182, hsa-miR-4640-3p, hsa-miR-2276, hsa-miR-4423-5p, hsa-miR-2277-3p, hsa-miR-513c-5p, hsa-miR-1250, hsa-miR-18a-3p, hsa-miR-505-5p, hsa-miR-138-2-3p, hsa-miR-548ah-3p, hsa-miR-4677-3p hsa-miR-1226-3p, hsa-miR-193b-5p, and hsa-miR-18b-5p.

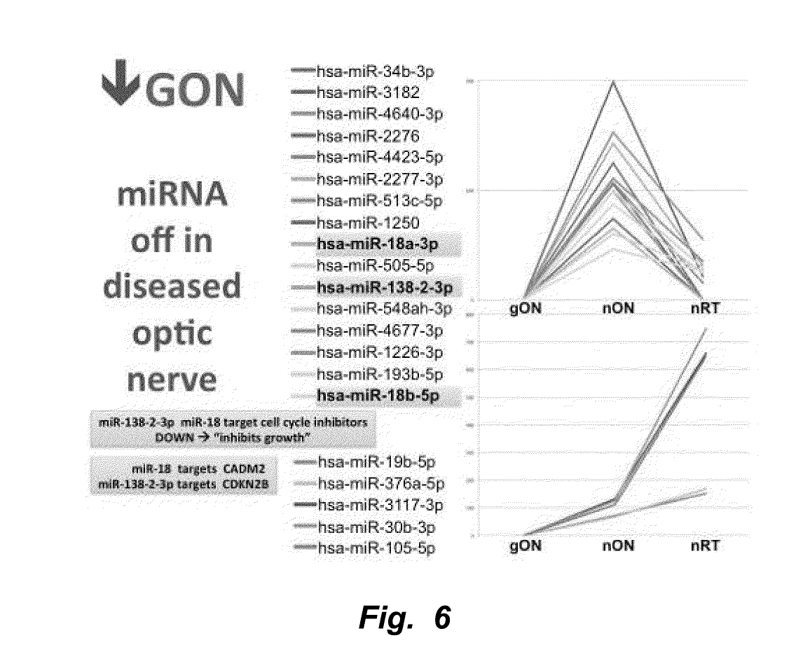

### DETAILED DESCRIPTION

### 1. Introduction

Provided herein in some embodiments are methods of identification of disease-associated genome variants in coding or regulatory regions of genes. The methods are exemplified in a preferred embodiment by the identification of genes that are associated with and/or promote onset or progression of a type of primary open-angle glaucoma. Other methods such as predictive, diagnostic, prognostic, and therapeutic methods are also provided herein.

The methods are based, in part, on the definition and use of a logic-based method to rank variants and genes based on clinical properties of disease. The methods are exemplified by application to variants from a cohort of patients with primary open angle glaucoma (POAG) and with elevated eye pressure, the method revealed 140 genes with variants over-represented in this disease in this embodiment. Genes were further ranked within the method based on gene expression patterns in tissues relevant to the disease process, which in the case of POAG can be retina, optic disk, optic nerve, ciliary body, choroid, trabecular meshwork, iris, sclera, and lamina cribrosa. Additional genes associated with the ranked genes were identified within the method as potential regulators of RNA and protein expression levels whose regulatory performance is disrupted or altered by highly ranked variants.

The method implements technical and clinical filters that reflect occurrence of disease in general populations. These filter reduced thousands of potential variants to under 150 for the preferred embodiment. The method incorporates gene expression information from tissues relevant to disease to refine ranked genes. The method incorporates information about potential microRNA, DNA-binding protein, and RNA-binding protein regulators of genes identified by the clinical ranking parameters.

The methods have been implemented as a body of software code written in Perl and other scripting languages, and applied to compare variations from a disease patient cohort (e.g., primary open angle glaucoma or POAG) with two publicly available datasets, e.g., 1000 Genomes (1000genomes.org) and the Exome Sequencing Project (evs.gs.washington.edu/EVS/) dataset. Other data sets can be used.

The genes identified by the analysis are potential targets or members of cellular pathways or processes that may be effective therapeutic targets in treating or curing the disease of interest (e.g., POAG). More particularly, disease onset, progression, severity, and/or recurrence can be addressed. Currently, for example, there is no cure for POAG and the only treatment is reduction of pressure in the eye to slow disease progression. Many variants found are in regulatory regions of genes and may control production of mRNA and/or protein. Molecules that bind to DNA or RNA at sites disrupted or altered by variants are further therapeutic targets.

The various embodiments described herein provide numerous, and in many cases surprising, advantages. For example, a key advantage in at least some embodiments is that a patient can receive earlier treatment for the disease such as POAG by use of the methods, screenings, and predictions described herein. Another key advantage in at least some embodiments is that a patient can receive more personalized or particular treatment for the disease such as POAG by use of the methods, screenings, and predictions described herein.

Moreover, despite the knowledge in the art, numerous surprising results were found throughout the presently described and claimed methodologies. For example, it was found that variant sites were concentrated in introns compared to coding regions.

In addition, it was found that the vast majority of the genes found in the glaucoma effort were not previously associated with glaucoma. However, for at least some of them, their functions within cells are in cellular processes related to glaucoma, e.g., genes involved in cell cycle, neural development and axon guidance, and inflammation.

In general, with the inventive filtering tool, the medical community is provided with a method to identify the genetic changes in a genome that are associated with a disease state, where those changes are not findable by standard GWAS or exome analysis methods. The newly identified sites provide a new patient management tool.

In addition, the approach described and claimed herein for glaucoma did find several genes previously associated with glaucoma, which puts new focus on those genes. Within those genes, the approach found sites that were not previously found in other studies because those studies focused on marker sites, whereas the presently described and claimed methods focus on finding causal sites inside the genes. Even further, it was found that frequencies of sites associated with glaucoma varied in frequency in the general population from very rare at <0.01 to very common at nearly 0.50.

Moreover, a list of genes was generated with their expression levels in tissues involved in the disease from human donor eyes. It was surprising to find genes and microRNAs that were differentially expressed across optic nerve, optic disc, retina, ciliary body, and trabecular meshwork tissues, and further were differentially expressed in tissues from eyes with disease compared with normal.

Also, microRNAs in optic nerve differed from microRNAs in retina and even optic disc. This was a large surprise because the optic nerve comprises axons of retinal ganglion cells whose nucleii are within the retina.

Hence, the technical effects of the claimed methodologies were clear, useful, and unexpected. Additional aspects of these technical effects are noted. For example, the elimination of false positive variants through direct genome sequence analysis of the region around the site early in the filtering steps is new and inventive.

Also important is the application of the clinical motivation to winnow sites of clinical utility. This led to filters that are more strict than have been used before (e.g., required ≧0.10 allele frequency difference between patients and general populations).

In addition, because the resulting sites passed clinical utility thresholds, they can be used directly for biomarker tests. The odds ratios of each final site, calculated after the direct filters were applied, range from 2 to 95. Their relative risk score ranges from 1.5 to 69. These are enormous and thus have much great clinical utility than glaucoma-associated sites found by others through GWAS with odds ratios of 1.1-1.4.

Furthermore, in the preferred embodiment, the patient frequency of each final site ranges from 0.18 to 0.98 with an average of 0.55. That is, large numbers of the HPG patients in which an allele was measured harbored each variant allele. The final sites are thus worth a clinician's time to consider and use in planning a patient's treatment.

In the preferred embodiments, human donor eyes were sought herein to gather RNA expression data for assessing sites found through our analysis. Surgical skill is required for the fine dissection of ocular tissues to find and harvest distinct tissues, e.g., optic nerve vs. optic disk, optic disk vs. retina and trabecular meshwork vs. iris and choroid. In addition, computational skill is required to analyze and interpret sequence reads obtained from tissues RNA and note differential expression of genes and microRNAs that control availability of those genes to make protein. The complementary and necessary surgical and computational skill resulted in assembly of a glaucoma-specific gene expression catalog which is and will continue to be a critical component to assess variants over-represented in HPG patients.

Some additional aspects of various embodiments are described, particularly with respect to prior art GWAS approaches, and citing eight references below. Standard approaches to genome-wide association used in the past and present apply a platform (e.g., Illumina 660 genotyping array, Illumina, San Diego, Calif.) to identify “marker” variants genome-wide in a large number of patients with disease (cases) and people confirmed not to have disease, often matched for attributes such as age (controls). Chichon et al provide a review of methods and their discovery power [15] Only variant sites measured in most cases and controls (e.g., 95% of both) are kept for analysis. After genotyping, the group of patients are checked for relatives (e.g., brother and sister in the patient cohort), repeated patients (e.g., a patient who moved from one study center to another), and population stratification (e.g., a number of patients with Mexican ancestry among Caucasian patients recruited from a southern state). Population features are corrected by eliminating subjects from the cohort or applying statistical corrections. Statistical tests are then applied to generate a p-value for each marker variant, and p-values less than 10⁻⁸ are considered to have “genome-wide significance” since the number of marker sites tested is generally on the order of 1 million (false discovery rate 0.05: 0.05/1M=10⁻⁸).

This procedure generates a list of markers that each point to a nearest gene or genes. Each plurality of markers near a given gene are subjected to additional statistical analysis and identify the gene as associated with disease. As multiple studies of the same disease are published, meta-analysis can be performed in which case cohorts are combined as are control cohorts; the larger numbers of cases and controls confer additional discovery power.

The following are some elements and considerations to these approaches: (1) Markers are chosen for the measurement platform to cover the genome evenly and completely. They do not indicate cause. (2) Markers may be over- or under-represented in the cases. Under-representation (Odds Ratio (OR)<1) indicates causal variant is likely to be nearby and over-represented in patients by virtue of being on a different version of the gene, i.e., a different haplotype. (3) Measured markers are restricted to known variants and may be restricted to those with general population frequency ≧0.05, depending on the platform. So variants rare in the population remain unmeasured. They can be inferred through statistical analysis of deeply sequenced genomes from general populations and assessing local recurring combinations of markers (a process called imputation). [16] (4) If the platform measures rarer variants with frequency ≦0.05, larger numbers of cases and controls are required to achieve p-values below 10⁻⁸ [17] (5) Sites associated with disease through GWAS generally explain disease in a small fraction of cases, e.g., 2%-4%. [18] (6) Meta-analysis requires harmonizing multiple datasets where genotypes were measured on different platforms. This reduces the sites measured and requires imputation for sites measured on one study's platform but not another's, which introduces uncertainty about measurements. See [19] as a comprehensive meta-analysis example.

Some additional description is provided of genome sequencing by short reads which provides more context for the various embodiments described herein. Genome sequencing aims to identify variants in a person's genome through direct DNA sequencing and assembly of DNA reads into contiguous stretches. [20] Genome sequencing can be expensive. For example, short-read sequencing with paired reads of length 10 bases (2×100) requires ˜480 million read-pairs for 30× coverage (30*3.2B/200=480M); at a cost of $1,250 per 200M read-pairs, a 30× genome is $3,000 plus costs for sample handling labor.

Some considerations of this include: (1) 30× coverage leaves random areas sparsely covered; so 100× is generally used for clinical purposes, more than tripling the cost to $10,000. (2) Rearrangements and repeats are more numerous between genes and make data analysis for variant discovery more complex.

A brief description of standard exome approaches to finding variants causing disease is provided. Exome sequencing uses DNA capture technology to sequence only the parts of genes that make molecules used in cells, e.g., exons that are protein coding or generate functional non-coding RNAs after an RNA transcribed from the genome has been spliced. [21] Captured exonic DNA is sequenced and mapped to a reference genome to find differences between a person's genome and the reference. The resulting variants may be causal of disease and are subjected to filtering to identify causal variants. Standard filters reject intronic and intergenic sites as off-target. Successful exome searches have focused on novel variants new in a small number (e.g., 10) patients with disease, as in [22]. Attempts to use large numbers of patients' exomes to associate variants with disease have failed to yield results.

Some considerations for this include: (1) Standard statistical treatments require variants to be measured in most cases and controls, but exome sequencing is a random capture process. So analyzable regions are restricted to those that are reliably captured, typically within or very near exons. (2) Standard variant callers require 10× coverage of a variant site to minimize false positive variant calls. Even so, false positives occur because of properties of the genome, e.g., tandem repeats or 2 or 3 nucleotides (e.g., CAGCAGCAG . . . ) or regions rich in G+C.

Clearly, a physician treating patients needs clear, causal information that applies to a given patient. Various embodiments described herein are designed to identify clinically useful variants through a novel evaluation process. Clinical utility of variants identified as associated with disease drive the invented process.

For example, one advantage for at least some embodiments is that every variant detected in one or more patients is considered for disease association. In contrast, standard GWAS or exome analysis requires variant alleles to be found in a larger number of patients.

Another advantage for at least some embodiments is that statistical analysis is applied to sites observed in 25 or more patients, and each site is statistically tested based on its number of observations in the patient cohort. In contrast, standard GWAS methods require uniform numbers of observations for all sites tested, e.g., measurement in 95% of cases and controls.

Furthermore, another advantage for at least some embodiments is that frequencies calculated from patients are compared to more than one available reference population. In the example, frequencies measured in HPG patients are compared with 1000 Genomes, Phase 1, since it is the most broadly used in the community, and then against the more recent release 1000 Genomes, Phase 3, with restriction to the subset of subjects of similar ancestry, and then against the Exome Sequencing Project, again with restriction to similar ancestry. In contrast, standard GWAS uses control cohorts measured along with the case cohorts; GWAS meta-analysis combines case cohorts for multiple studies into one and compares with one combined control cohort.

Moreover, another advantage for at least some embodiments is that since the majority of sites measured in patients are concordant with general population frequencies, outliers are identified in two steps that are clinically motivated rather than statistically motivated.

In a first step, an absolute difference threshold is applied (≧0.10, in example). This recognizes the clinical motivation that in a well-phenotyped patient population that harbors genetic causes of disease, the disease-causing variants should be vastly higher than general populations. This restricts variants to those that will be clinically significant. This is in contrast to findings in GWAS studies where frequency deviations may be as small as 2% but have strong p-values. By restricted sites to those with large differences, final sites will be clinically significant.

In a second step, an odds-ratio and confidence interval are calculated, and the confidence interval lower bound must be above 1.0. Clinicians need strong, clear indications of risk for disease and avoid making treatment decisions based on low confidence data.

In contrast, GWAS and meta-analysis identify outliers based on p-values and genome-wide significance thresholds, thus accepting as disease-associated variants that do little to explain disease and with little or no clinical utility.

Another advantage of at least some embodiments is that false positives are minimized through a novel series of filters so that variant detection can be more sensitive. As a result, more variants, including many deep inside introns or upstream of genes in promoter regions can be considered for relationship to disease. Problematic variants are identified in two steps.

(a) First, false variants can emerge from the mapping process. Others have tried to improve mapping. Here, sources of mapping bias are identified directly and captured as two exclusion lists. These lists holds sites for which (i) the reference base is the minor allele in the reference genome used for mapping; and (ii) the alternate allele found in patients in also the minor allele in general populations. In the example, these two exclusion lists eliminated from further consideration 1,188,903 and 127,620 variant sites, respectively.

(b) Second, every candidate variant site is screened against a constructed list of sites genome-wide that have anomalies within the genome region. Such anomalies can introduce false positive variant calls. The approach here relies on three exclusion lists that were constructed to implement three sequence-based filters. These lists hold sites computed to occur within 100-200 bases with (i) GC/AT bias; (ii) replicates elsewhere in the genome; and (iii) tandemly repeated motifs. In the example, the exclusion lists were used to reject 77,149 sites within regions of GC/AT bias, 56,905 sites within sequences repeated elsewhere in the genome, and 124 sites with tandem repeats.

In contrast, standard exome methods simply do not filter variants directly based on genome sequence properties.

Another important point is that because problematic variants are filtered directly by direct analysis of genome sequence properties, false variants are minimized before any statistical tests are applied. This allows a lower threshold on the number of reads needed to call a variant. Where other exome interpretation approaches require a minimum of 10 reads, our approach requires a minimum of three. The further a variant is from the exome probes used for capture, the lower its coverage with reads. In the example, variants inside genes but as far as 10,000 bases from upstream or downstream of exons were considered for their disease-relatedness. Consequently, the final list of HPG variants includes a large number of intronic variants, which are missed entirely by standard exome analysis methods. In the example, the list of 932 variants remaining after step 15 contain only 75 sites present in the Exome Sequencing Project database, and the final list of 160 sites contains just 23 sites in ESP.

In contrast, GWAS studies are limited to sites represented on commercial genotyping platforms and do not include variants novel in a patient, and exome studies are limited to sites with uniformly deep coverage across the exome.

In addition, the focus here is on variants that cause chronic, systemic diseases in the general population at rates higher than, say, 1%, i.e., common diseases. Such variants are unlikely to be novel within patient populations. Otherwise the disease would be far less common. However, combinations of lower frequency variants may together explain disease across a patient population. Here, variants are considered for disease association regardless of their frequency in general populations, and all variants detected in patients are considered.

In contrast, GWAS studies are limited to sites represented on commercial platforms, and other exome studies have used approaches that focused on novel and rare variants.

### 2. Methods of Identifying Genes Causing Onset or Affecting Progression or Severity of Disease

Generally the source material sequences of use in the present methods have been sequenced with high fidelity, e.g., the sequences determined with 4 or fewer mismatches per 100 bases, e.g., with 4, 3 or 2 or fewer mismatches per 100 bases.

Table 2 provides a summary of steps that can be taken in the inventive methods for the preferred embodiment of POAG. One skilled in the art can vary the order of steps as needed for a particular application. One skilled in the art also can eliminate one or more steps as needed for a particular application. One or more technical, clinical, gene-based, and/or statistical constraints listed in Table 2 (e.g., for genes associated with and/or causative of HPG) are applied for the selection of genes associated with or causative of a disease condition. First, sites are counted if observed as variant either from a reference genome or from other patients. Second, sites are evaluated if reported in a publicly available genome dataset, e.g., 1000G, the primary comparison population. Third, sites are restricted to those observed as variant in 3 or more patients. Fourth, to limit false positive effects due to reference bias during mapping, sites are excluded if the base in the hg19 reference genome was the minor allele base in 1000G. Fifth, sites are included only if the alternate allele remained the minor allele in general populations of similar ethnic descent as the patient cohort. Sixth, sites found to have more than one alternative base are set aside for future consideration. Seventh, eighth and ninth, sites are restricted to those in genome regions with balanced G+C and A+T content; located outside low complexity regions; and located in genome regions without nearly identical, e.g., within 95% identity, paralogs elsewhere. Tenth, any sites located on the X-chromosome or the Y-chromosome are unlikely to contribute to a target disease (e.g., high pressure glaucoma) unless the disease has a clear gender predilection, and therefore can be excluded (e.g., limit selection to genes expressed from chromosomes 1-22). See, Ederer, et al., 1994 [23]. Thus sites on the X and Y chromosomes are excluded from further analysis.

Next, three constraints based on clinical criteria are applied as prerequisites for association with disease. Eleventh, a SNP site must be observed in enough patients to calculate its importance in disease. Because sequencing does not always capture a given site in all samples, the denominator for frequency calculation for a SNP site becomes twice the number of samples with reads at that site. In varying embodiments, sites are excluded from consideration if they are measured in fewer than 25 patients. Twelfth, a genomic aberration is not likely to be important as a primary cause of a target disease (e.g., high pressure glaucoma) if it occurs with frequency close to that in the normal population. In varying embodiments, sites with patient frequencies within measurement error, e.g., 0.05, of the 1000 Genomes Phase 1 general population frequency are set aside, as are sites with patient frequencies within measurement error of the European subset of the 1000 Genomes Phase 3 subjects. Likewise, sites with patient frequencies within measurement error of the European subset of the Exome Sequencing Project (ESP) are set aside. Thirteenth, in varying embodiments, SNP sites with allele frequencies of greater than the prevalence of the target disease (e.g., high pressure glaucoma, with occurs in about 2 to 4% of the adult general population) in any adult general population used for comparison are excluded. Further, in varying embodiments, sites are kept if their patient allele frequency substantially exceeds general population frequency, e.g., by 0.10 or greater in any adult general population used for comparison.

Next, two gene-base criteria are applied. Fourteenth, sites outside of a gene or regulatory regions influencing its expression as RNA or protein are excluded from further analysis as off target. Fifteenth, sites within or near genes expressed in tissues relevant to disease are retained.

Next, three statistical criteria are applied. Sixteenth, odds ratio and confidence interval are calculated for each site based on number of patients in whom the site was measured, the number of alternate alleles observed, and the number of measured and alternate alleles in the 1000G Phase 3 database. Sites with a 95% odds ratio confidence interval lower bound above 1.0 are retained. Seventeenth, sites are further retained if their frequency in patients is above a statistical fit of a line to datapoints where X is reference general population frequency and y is patient frequency. In some embodiments, the fit is performed with a least square linear estimate function. Eighteenth, a 2×2 statistical test is applied to obtain p-values. In some embodiments, Fisher's Exact Test is used. Sites are then grouped by the number of patients, N, in which they are measured, and a significance threshold is calculated for each measurement group. In some embodiments, the Bonferroni formula (0.05/N) is used to calculate the threshold maximum p-value to determine significance under multiple testing. SNP sites passing these constraints indicate genes important in the target disease (e.g., high pressure glaucoma, ocular diseases and disorders, Alzheimer's, Parkinson's, Prion Disease (PRNP) and other misfolded protein diseases).

Analysis of the sequencing data and the diagnosis derived therefrom can be readily performed using various computer executed algorithms and programs, using appropriate software and hardware available to one skilled in the art. Therefore, certain embodiments employ processes involving data stored in or transferred through one or more computer systems or other processing systems. Embodiments disclosed herein also relate to apparatus for performing these operations. This apparatus may be specially constructed for the required purposes, or it may be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel. A processor or group of processors for performing the methods described herein may be of various types including microcontrollers and microprocessors such as programmable devices (e.g., CPLDs and FPGAs) and non-programmable devices such as gate array ASICs or general purpose microprocessors.

In addition, certain embodiments relate to tangible and/or non-transitory computer readable media or computer program products that include program instructions and/or data (including data structures) for performing various computer-implemented operations. See, for example, WO 2014/080323 for use of non-transitory computer readable or storage media in the genomic context. Examples of computer-readable media include, but are not limited to, semiconductor memory devices, magnetic media such as disk drives, magnetic tape, optical media such as CDs, magneto-optical media, and hardware devices that are specially configured to store and perform program instructions, such as read-only memory devices (ROM) and random access memory (RAM). The computer readable media may be directly controlled by an end user or the media may be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that may be executed by the computer using an interpreter.

In various embodiments, the data or information employed in the disclosed methods and apparatus is provided in an electronic format. Such data or information may include reads and tags derived from a nucleic acid sample, counts or densities of such tags that align with particular regions of a reference sequence (e.g., that align to a chromosome or chromosome segment), reference sequences (including reference sequences providing solely or primarily polymorphisms), counseling recommendations, diagnoses, and the like. As used herein, data or other information provided in electronic format is available for storage on a machine and transmission between machines. Conventionally, data in electronic format is provided digitally and may be stored as bits and/or bytes in various data structures, lists, databases, etc. The data may be embodied electronically, optically, etc.

### 3. Identified Biomarkers Causing Onset or Affecting Progression of Primary Open Angle Glaucoma (POAG) or High Pressure Glaucoma (HPG)

For the preferred embodiment, biomarkers, including genes and microRNAs, determined to be associated with and/or causative of POAG and/or HPG are provided in Tables 4, 5, and 6. In Table 4, the alternative (ALT) allele is associated with disease. Tables 5 and 6 summarize microRNAs that are overexpressed or underexpressed in tissues from patients having POAG and/or HPG. In varying embodiments, expression of any of the listed biomarkers in Tables 4, 5, and 6 can be determined in the various ocular tissues, including without limitation trabecular meshwork (TM), ciliary body (CB), choroid (CH), optic disk (OD), optic nerve (ON) and retina (RT). Methods known in the art can be used to determine expression levels.

The POAG/HPG associative and/or causative genes discovered herein (e.g., as summarized in Tables 4, 5, and 6) can be evaluated and/or monitored with genes known to be associated with and/or causative of glaucoma and/or other eye diseases. Prior genome-wide association and linkage-based studies have identified loci with contribution to glaucoma including myocilin, CYP1B1, optineurin, WDR36, TBK1, TBK2, and GALC. Loci contributing to POAG found through GWAS include TMCO1, CAV1/CAV2, CDKN2B-AS1, SIX1/SIX6, TXNRD2, ATXN2, FOXC1, an 8q22 intergenic region, and GAS7. Loci associated with optic disk area, a phenotype relevant to POAG include ATOH7/PBLD, CDC7/TGFBR3, and SALL1. Loci associated with vertical cup to disk ratio (CDR), a useful measurement to monitor progression of optic neuropathy in POAG, include SCYL1/LTBP3, CHEK2, ATOH7, DCLK1, SIX1/SIX6, CDKN2A/B, and CDKN2B-AS1. Several genes are strongly associated with central corneal thickness (CCT), including FOXO1, COL5A1, ZNF469, AKAP13, AVGR8, and COL8A2; however, recent genetic studies indicate CCT may not be directly associated with susceptibility to POAG. Molecular studies of differential gene expression in tissues relevant to glaucoma revealed genes up- or down-regulated in trabecular meshwork, lamina cribrosa, and optic nerve head astrocytes from glaucomatous eyes compared to eyes without disease. In the latter, among 183 up-regulated and 220 down-regulated genes, a number of genes previously studied in eye disease and development had notable differences in glaucomatous compared to normal astrocytes, including TGFB1, SPARC, POSTN, THBS1, CRTL-1, COL1A1, COL5A1, COL11A1 (up) and FBLN1, DCN, COL18A1 (down). Likewise, studies of differential expression in glaucomatous trabecular meshwork, the eye tissue involved in aqueous outflow, revealed additional genes of interest, as did studies of lamina cribrosa from glaucomatous eyes. The OMIM database of diseases and genes maintained at NCBI aims to provide a comprehensive list of disease-related genes for all human diseases. OMIM reports nine genes directly related to glaucoma. These include five additional genes, FOXC1, LTBP2, NTF4, OPA1, and SBF2, and four genes listed above, CYP1B1, MYOC, OPTN, WDR36. OMIM lists 29 genes indirectly related to glaucoma: APOE, BEST1, BMP4, CA12, CANT1, CNTNAP2, CRB1, EPO, FOXE3, FOXL2, GJA1, GLIS3, ISPD, LMX1B, LOXL1, MTHFR, PAX6, PEX5, PITX2, PITX3, POMT1, RPS19, RRM2B, SLC4A4, TDRD7, TGFB2, TNF, and TTR as well as TMCO1 listed above. The National Eye Institute's EyeGene project maintains a database of genes involved in any eye disease and their variants causing disease. EyeGene reports genes for eye diseases ranging in onset from congenital to late-age, including microphthalmia, retinal degeneration, macular degeneration and various forms of glaucoma. See also, genes discussed in van Koolwijk, et al., 2013 [24], Burdon et al., 2012 [25], Allingham, et al., 2009 [26]. One skilled in the art can combine prior art knowledge with the inventive features described and claimed herein to address disease.

### 4. Predicting Onset and/or Progression and/or Severity and/or Recurrence of Disease

Another important aspect is a method for predicting onset and/or progression and/or severity and/or recurrence of disease (e.g, primary open angle glaucoma (POAG)) in a subject, the method including receiving allelic information and/or expression levels of a collection of signature biomarkers from a biological sample taken from the subject suspected of developing or suffering a disease such as POAG, wherein said collection of signature biomarkers comprises one or more genes and/or microRNA selected from a group developed using the methods described herein.

One can then apply the allelic information and/or expression levels to a predictive model relating allelic information and/or expression levels of said collection of signature biomarkers with onset of POAG; and evaluate an output of said predictive model to predict onset of POAG in said individual.

One can then also apply the allelic information and/or expression levels to a predictive model relating allelic information and/or expression levels of said collection of signature biomarkers with progression of POAG; and evaluate an output of said predictive model to predict progression of POAG in said individual.

One can then also apply the allelic information and/or expression levels to a predictive model relating allelic information and/or expression levels of said collection of signature biomarkers with severity of POAG; and evaluate an output of said predictive model to predict severity of POAG in said individual.

One can then also apply the allelic information and/or expression levels to a predictive model relating allelic information and/or expression levels of said collection of signature biomarkers with recurrence of POAG; and evaluate an output of said predictive model to predict recurrence of POAG in said individual.

Combinations of onset, progression, severity, and recurrence can be carried out for a particular patient and used for further prognostic, diagnostic, and/or therapeutic steps. Kits can be used for testing of subjects.

### EXAMPLES

The following examples are offered to illustrate, but not to limit the claimed invention.

### Example 1

### Disease-Associated Variants in Coding and Regulatory Regions Revealed by Exome Sequencing in High-Pressure Open-Angle Glaucoma Patients

In glaucoma, progressive optic nerve degeneration can lead to irreversible vision impairment and eventual blindness, despite treatment. Genetic causes and influences are not yet clear in primary open angle glaucoma (POAG), the most prevalent form of the disease in North America, Europe, and several other parts of the world. The genetics of POAG are complex; to date, no single causative genomic variant has been established as causing the disease. We have studied the genomes of 295 high-pressure POAG (HPG) patients and compared findings with general population observations found in the 1000 Genomes and the Exome Sequencing Projects. We have identified 160 genome polymorphisms greatly overrepresented in HPG patients compared with general populations. These changes are located in coding and regulatory regions of 140 genes and implicate these genes in HPG. The variants implicating these genes are potential causative factors. For all genes, mRNA expression was detected in ocular tissues. Five of the 140 were already associated with POAG or its phenotypic risk factors. The remaining 135 genes are newly implicated in HPG. These genes and their variants complement a growing list of genes involved in glaucoma found through linkage, genome wide association, and other studies. This opens new avenues for investigation into the genetic, molecular, and biochemical mechanisms of this disease.

### Methods:

Inclusion and Exclusion Criteria.

The DNA samples for this study are a subset of the de-identified samples from patients enrolled in the NEIGHBOR GWAS. Patients with primary open angle glaucoma (POAG) were enrolled in NEIGHBOR after confirmation of reliable visual field (VF) tests with characteristic defects on two or more tests, or with a single qualifying VF test accompanied by a vertical cup-disc ratio of 0.7 or more in at least one eye. Examination of the ocular anterior segment disclosed no signs of secondary causes for elevated IOP. The approach to the filtration structures in the anterior chamber angle was wide open on gonioscopic examination. All patients selected for the present study had a documented, confirmed history of IOP ≧22 mm Hg and were classified as HPG [8,27]. (Table 1. Demographics) Each NEIGHBOR-enrolled patient gave informed consent at their site of ophthalmic care to donate a blood sample for glaucoma genome investigations. Collaborating physicians obtained blood samples at the site of care and submitted them to NEIGHBOR for DNA preparation, storage, and study-related investigations.

DNA Target Enrichment and Sequencing.

DNA samples from 295 patients were indexed and prepared for deep sequencing on the Illumina HiSeq2000 instrument (Illumina, San Diego, Calif., USA). Two indexed samples at a time were pooled, and DNA regions that code for proteins genome-wide were enriched using Nimblegen SeqCap EZ version 2 (204 samples) or version 3 (109 samples) (Roche Nimblegen, Madison, Wis., USA). Paired DNA sequences (readpairs) of length 100 bases (2×100) were determined for enriched DNA to generate a minimum of 50 million readpairs per sample. The hg19 reference genome 14 contains 21,210 genes with HUGO identifiers and 464,698 exons annotated in the Refseq database at NCBI. The Nimblegen V2 probes were designed to cover 44,070,352 bases in 392,771 Refseq exons and 18,804 genes with HUGO identifiers. The Nimblegen V3 probes were designed to cover an expanded target region with 64,148,113 bases in 410,269 exons and 19,721 genes.

Read Alignment.

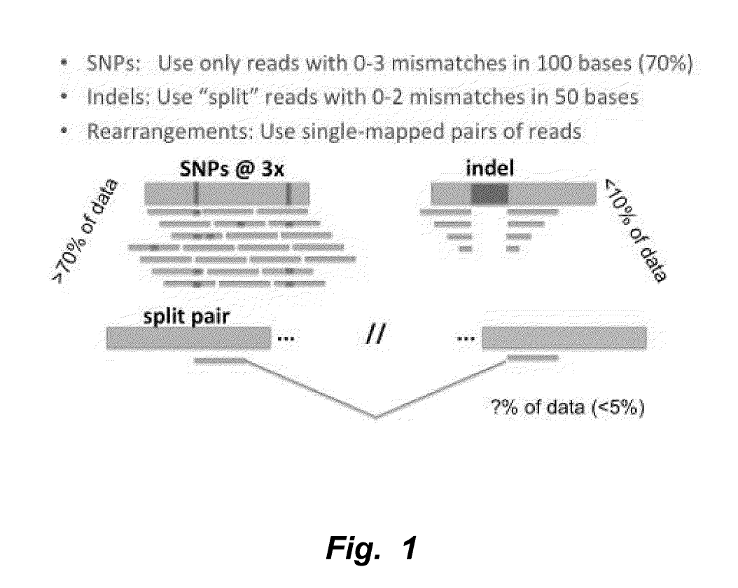

The sequence data analysis strategy was designed to minimize false positive observations and focus on SNP sites where nucleotides observed in the patient DNA had differences, either homozygous or heterozygous, from the human reference genome version hg19. Sequence data for human chromosomes 1 through 22, X, Y and mitochondria were downloaded from the UC Santa Cruz Genome Browser (http://genome.ucsc.edu) [28] and prepared as a target for mapping paired reads using the BowTie software [29]. Reads with more than three mismatches to the reference genome or with matches to more than one genome locus were set aside as unmapped for future detection of insertions, deletions and tandem repeat expansions. FIG. 1 illustrates the read mapping strategy. Mapped reads were converted from a text-based sequence alignment/map (SAM) format to a binary (BAM) format with Samtools [30].

Sequence Data Quality Filtering and Genotyping.

The BAM files for each sample were reviewed to determine whether reads were sufficient to determine genotypes at variant sites across the targeted capture regions. Any sample with insufficient breadth of coverage was excluded from further analysis. This yielded 295 samples with sufficient sequencing (Table 1). Each remaining BAM file was treated as follows: All sequence data were analyzed with respect to the forward strand of the hg19 reference genome. The Samtools “pileup” algorithm 16 was called to extract bases from reads at every sequenced site to produce a list of bases (“pileup”) and a consensus base at each site. Each pileup was separated into evidence agreeing with the hg19 reference base and evidence for an alternate base at that site. To call either a reference or alternate base as present in the patient genome, reads were required to be from both forward and reverse DNA strands, with at least three high quality reads per base for the genotype to be considered heterozygous (two or more differing nucleotides) or four high quality reads to be considered homozygous (two copies of one nucleotide). Further, for a heterozygous genotype, the ratio of reads supporting each nucleotide had to be between 0.5 and 2, indicating the reads were balanced between both chromosomes. If this analysis found evidence that supported either the hg19 reference or an alternate base yet did not meet the criteria for a call, the site was designated as “no call” for the sample, and the observation of the site in the patient flagged as “ambiguous”. For a given patient, sites with reads in other patients but no reads in this patient were designated as “no call” and flagged as “missed”. This process yielded an explicit genotype call, including flagged “no calls”, for every sample at every site sequenced in any patient.

TABLE 1
Demographics

| Variable¹ | Cases |
|---|---|
| Number | 295 |
| Female | 54% |
| Age (years), mean (SD) | 62 (±15) |
| IOP (mm Hg), mean (SD)² | 16 (±6) |
| CDR, mean (SD)² | 0.82 (±0.16) |
| POAG in 1° relatives | |
| Hx Obtained | 281 |
| Positive | 200 |
| Percent Positive | 71% |
| History of Diabetes | 9% |
| History of Hypertension | 43% |

¹Abbreviations: IOP = treated intraocular pressure, CDR = vertical cup to disc ratio, SD = standard deviation
²Means are mean of both eyes.

HPG Variant Identification and Annotation.

Genome sites from the 295 patients with sufficient sequence data and evidence of difference from hg19 reference were put into a Master Variant Table and submitted to the SeattleSeq Annotation server (available at snp.gs.washington.edu) [31]. The table included every site observed with an allele call different from the reference genome in at least one patient. SeattleSeq returned annotations for each site with gene names, dbSNP database identifiers for known SNPs, whether a SNP changes a protein amino acid, likely impact of the change on the protein using the PolyPhen2 and SIFT2 algorithms [32,33,34], distance to nearest exon-intron splice site, distance to stop codon for SNPs in untranslated regions, distance to nearest gene for intergenic SNPs, relative conservation of DNA around the SNP across mammalian genomes, and any known clinical or disease association. The annotations were added to the Master Variant Table to support further analysis and search for genes associated with HPG.

HPG Allele and Zygosity Frequencies.

Allele and zygosity frequencies were calculated for every site in the Master Variant Table based on the genotype calls for each patient sample. For each SNP site in chromosomes 1 to 22, the observed frequency for an alternate base (a) was determined as the number of heterozygous observations (het) plus twice the homozygous alternate base observations (hom) divided by twice the number of samples (n) that had a genotype call (including homozygous same as reference base) at that site, thus a=(het+2*hom)/2n. This allele frequency, a, became the basis for identification of SNP site alleles potentially overrepresented in HPG patients.

Comparison with 1000 Genome (1000G) and Exome Sequencing Project (ESP) Databases.

Comparisons between the HPG data and general population databases were based on the less frequent (minor) allele in the 1000G database for every SNP site identified in the 295 patients. Comparison tables were constructed from public variant tables downloaded from the 1000G server (1000genomes.org/data, ftp://ftp.1000genomes.ebi.ac.uk/vol1/ftp/, Phase1 Integrated Release Version3_{—}20120430 with 38,248,780 sites, including 14,675,062 sites with frequencies derived from European subpopulations; and Phase 3 Release 20130502 with over 79 million variants, including variants measured in 505 subjects of European descent) [35] and the ESP Exome Variant Server (evs.gs.washington.edu/EVS/, ESP6500 Version 2 with 3,688,361 sites) [36]. These tables include chromosome positions, allele bases, allele frequencies, and supporting information. The minor allele in the 1000G database was identified for every 1000G site. To limit false positives in our analysis due to reference bias inherent in the mapping process, we identified all sites where the hg19 reference base was the minor allele base in 1000G. The HPG and general population frequencies for the 1000G minor alleles were used in all further comparisons to identify sites of interest in HPG patients.

Application of Constraints.

Technical, clinical and statistical considerations allowed definition of constraints to apply to the SNP sites identified in the HPG patients. See Table 2. (Table 2, Abbreviations: HPG, high pressure primary open angle glaucoma, 1000G, 1000 Genomes Project; ESP, Exome Sequencing Project.)

TABLE 2

| | |
|---|---|
| Constraint | Sites |

a. Criteria for variant identification, exclusion, and inclusion

| | | | |
|---|---|---|---|
| 1 | Patient alleles different from reference genome are eligible for consideration. | Sites variant in 1+ patients | 4,267,157 |
| 2 | Public database variants have frequency and population information available for | Sites in 1000G Phase 1 | 4,032,533 |
| | comparisons. | | |
| 3 | Variants observed in 3+ unrelated patients are more reliable than those found in only 2 | Sites variant in 3+ patients | 2,748,984 |
| | or 1. | | |
| 4 | At some sites, reference genome has minor allele, which may cause “reference bias”. | Reference base is major allele | 1,560,081 |
| 5 | At some sites, general population minor allele is major allele in populations of | Alternate allele minor in 1000G Phase 3 | 1,432,461 |
| | European descent. | European population | |
| 6 | Sites with multiple alternate alleles are difficult to interpret reliably. | Only one alternate allele | 1,423,956 |
| 7 | Sites in regions with high G + C content or long single nucleotide repeats may be | Balanced G + C and A + T content | 1,350,492 |
| | sequencing anomalies. | | |
| 8 | Sites in regions within low complexity regions may be sequencing anomalies. | Located outside low complexity regions | 1,350,455 |
| 9 | Sites in regions with nearly identical copies elsewhere in the genome (≧95% identical) | Located in genes with no nearly identical | 1,302,588 |
| | may be mapping anomalies. | paralogs | |
| 10 | Chromosomes X and Y can be set aside when gender bias of disease is minimal. | Chromosomes 1-22 | 1,279,295 |

b. Prerequisites for association of variants with disease

| | | | |
|---|---|---|---|
| 11 | Observations in 25+ patients are needed to yield granularity of calculated | Observed in 25+ patients | 455,413 |
| | frequencies within ±0.02. | | |
| 12a | Alleles with frequencies within 0.05 in patients and general population are unlikely | 1000G Phase 1 general population and | 40,860 |
| | to explain disease. | patients differ by more than 0.05 | |
| 12b | 1000G Phase 1 general population frequencies differ from Phase 3 European | 1000G Phase 3 European population and | 8,336 |
| | frequencies for some sites. | patients differ by more than 0.05 | |
| 12c | ESP population frequencies differ from 1000G frequencies for some sites. | If in ESP, ESP European population and | 7,985 |
| | | patients differ by more than 0.05 | |
| 13 | Alleles explaining disease will be overrepresented in patients compared to general | Patient allele frequency and general | 2,235 |
| | populations. | populations differ by more than 0.10 | |

c. Gene-based criteria

| | | | |
|---|---|---|---|
| 14 | Intergenic sites are off-target. | Sites located within genes or their | 1,408 |
| | | regulatory regions | |
| 15 | Sites in genes expressed in relevant tissues are more likely to impact disease. | Gene expression in relevant tissues | 932 |

d. Statistical criteria

| | | | |
|---|---|---|---|
| 16 | Alleles with odds ratio (OR) 95% confidence interval overlapping 1.00 are less | OR confidence interval above 1.00 | 506 |
| | likely associated with disease. | | |
| 17 | Least squares linear regression fit of data distinguishes alleles most likely | HPG frequency exceeds least squares | 199 |
| | associated with disease. | linear regression fit of data. | |
| 18 | Fisher's Exact Test p-values of ≦0.05 must remain significant after Bonferroni | P-value less than Bonferroni correction | 160 |
| | correction for multiple tests. | | |

Ten constraints for variant identification, exclusion and inclusion were applied as follows. First, sites were counted if observed as variant either from a reference genome or from other patients. Second, sites were evaluated if reported in a publicly available genome dataset, e.g., 1000G, the primary comparison population. Third, sites were restricted to those observed as variant in 3 or more patients. Fourth, to limit false positive effects due to reference bias during mapping, sites were excluded if the base in the hg19 reference genome was the minor allele base in 1000G. Fifth, sites were included only if the alternate allele remained the minor allele in general populations of similar ethnic descent as the patient cohort. Sixth, sites found to have more than one alternative base were set aside for future consideration. Seventh, eighth and ninth, sites were restricted to those in genome regions with balanced G+C and A+T content; located outside low complexity regions; and located in genome regions without nearly identical, e.g., within 95% identity, paralogs. Tenth, any sites located on the X-chromosome are unlikely to contribute to HPG because this disease has no clear gender predilection; X and Y chromosome sites were excluded.

Next, three constraints based on clinical criteria were applied as prerequisites for association with disease. Eleventh, a SNP site must be observed in enough patients to calculate its importance in disease. Because sequencing does not always capture a given site in all samples, the denominator for frequency calculation for a SNP site becomes twice the number of samples with reads at that site. Sites were excluded from consideration if they were measured in fewer than 25 patients. Twelfth, sites with patient frequencies within measurement error, e.g., 0.05, of the 1000 Genomes Phase 1 general population frequency were set aside, as were sites with patient frequencies within measurement error of the European subset of the 1000 Genomes Phase 3 subjects. Likewise, sites with patient frequencies within measurement error of the European subset of the Exome Sequencing Project (ESP) were set aside. Thirteenth, since POAG occurs in about 2 to 4% of the adult general population, sites were kept if their patient allele frequency substantially exceeded general population, e.g., by 0.10 or greater in a comparison adult general population.

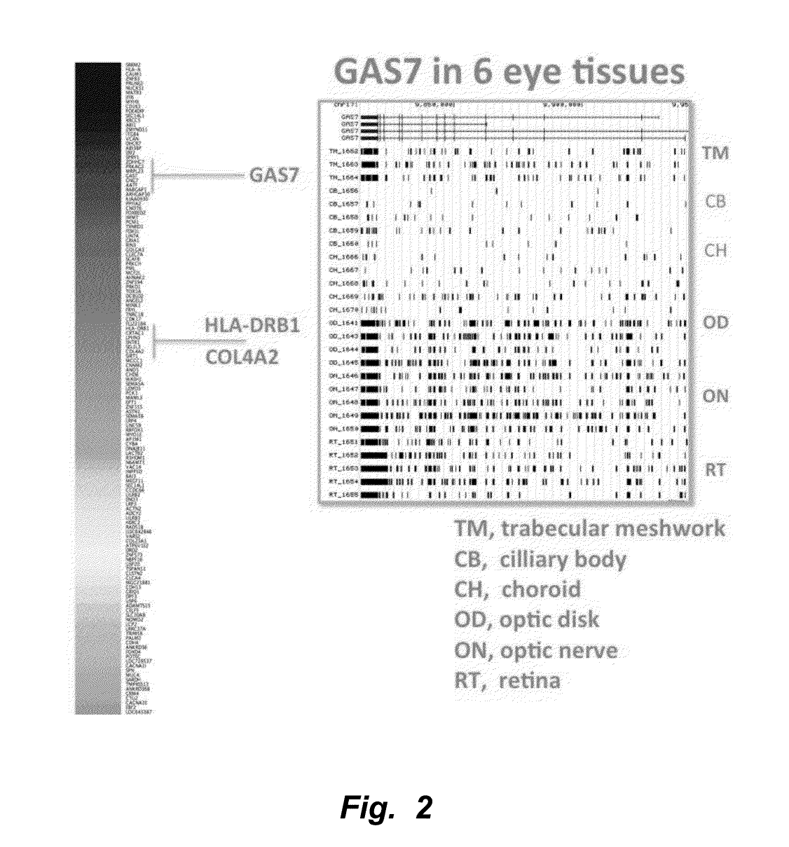

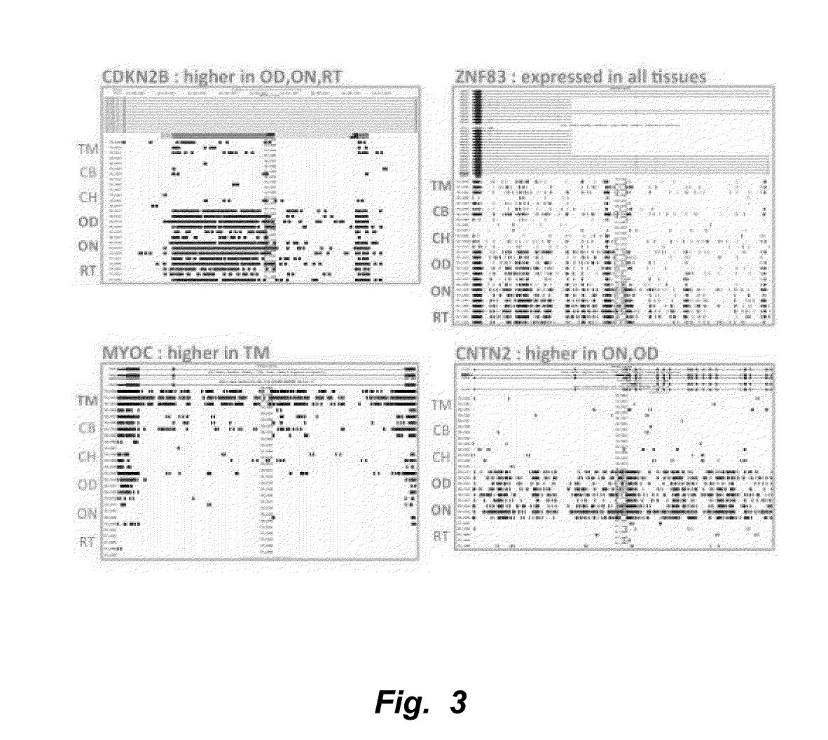

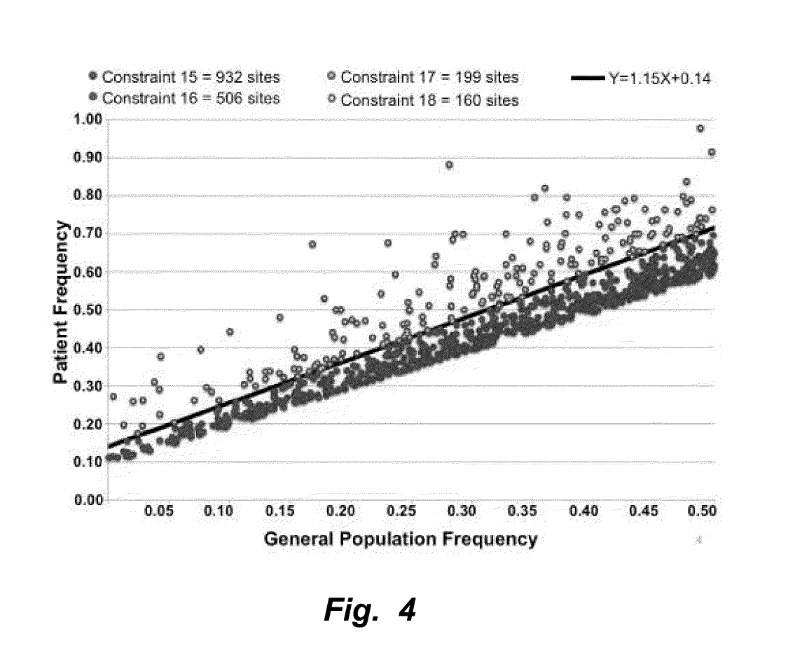

Next, two gene-base criteria were applied. Fourteenth, sites outside of a gene or regulatory regions influencing its expression as RNA or protein were excluded from further analysis as off target. Fifteenth, sites within or near genes expressed in tissues relevant to disease were retained. FIGS. 2-4 illustrate gene expression in ocular tissues.

Next, three statistical criteria were applied. Sixteenth, odds ratio and confidence interval were calculated for each site based on number of patients in whom the site was measured, the number of alternate alleles observed, and the number of measured and alternate alleles in the 1000G Phase 3 database. Sites with a 95% odds ratio confidence interval lower bound above 1.0 were retained. Seventeenth, sites were further retained if their frequency in patients exceeded a least squares linear regression fit of datapoints where X was reference general population frequency and Y was patient frequency. Eighteenth, a 2×2 Fishers Exact Test was applied to obtain p-values. Sites were grouped by the number of patients, N, in which they had been measured, and a significance threshold was calculated for each measurement group using the Bonferroni formula (0.05/N) to correct for multiple testing. SNP sites passing these constraints indicate genes important in HPG.

Gene Expression in Ocular Tissues.

To measure gene expression in six ocular tissues (retrobulbar optic nerve, optic disc, retina, choroid, ciliary body, and trabecular meshwork), we performed whole transcriptome sequencing (RNA-seq) of tissues dissected from 5 fresh donor human autopsy eyes, two with history of primary open angle glaucoma and three without. RNA was extracted, fragmented to ˜200 bp (basepairs), ligated with Adaptor Mix, converted to cDNA with ArrayScript Reverse Transcriptase (Ambion), size selected (˜200 bp) by gel electrophoresis, and PCR amplified with adaptor primers. Deep sequencing was done on an Illumina HiSeq 2000. Differential gene expression analysis was done with TopHat and CuffLinks. See, e.g., Trapnell, et al., Nat Biotechnol. (2013) 31(1):46-53. For each tissue, reads were pooled and mapped to the hg19 reference genome. Reads per kilobase of exon per million mapped reads (RPKM) were calculated for each gene and used as an estimate of expression.

### Results:

Demographics.

The genomes of 295 patients with HPG were the focus of this study (Table 1). Females constituted 54%. The mean age at diagnosis was 62 (±15 SD, range 30 to 94) years. Treated mean IOP at the time of blood sampling was 16 (±6, range 4 to 32) mmHg. The mean of the vertical cup-disc ratio was 0.82 (±0.16, range 0.30 to 1.00). There was a self-reported history of open-angle glaucoma in a 1^{st} degree relative in 69 percent, of Type 2 diabetes in 9 percent and a history of hypertension in 43 percent of patients in the present study.

HPG Target Enriched Sequencing, Alignment, and Annotation.

Of the 295 samples analyzed, 105 were captured with Nimblegen V3 and 190 with Nimblegen V2.

Identification of Glaucoma-Related SNP Sites and Genes.

The initial review of the sequencing data disclosed 4,267,157 sites in the HPG patients that differed from the hg19 reference genome in any patient. A series of constraints were applied to identify the SNP sites in or near exons wherein an alternate allele was over-represented in HPG patients.

First, a series of ten constraints identified, included or excluded variants. Of the sequenced sites, 4,267,157 were variant in 1 or more HPG patients (Constraint 1). This number fell to 4,032,533 upon limiting to sites found in the 1000G public database (Constraint 2). Of these, 2,748,984 were variant in 3 or more HPG patients (Constraint 3). Some of the sites in the reference genome had the minor allele in the comparison database, 1000G, potentially causing reference bias during analysis, and were eliminated from consideration; 1,560,081 sites had the major allele as the reference base (Constraint 4). For some sites, the alternate allele, although minor in the 1000G Phase 1 generation population, became the major allele in the European population and were eliminated, yielding 1,432,461 sites (Constraint 5). Next, 1,423,956 of the sites remaining after the previous constraint had no more than one alternate allele in the HPG patients (Constraint 6). Of these, 1,350,492 had balanced G+C content (Constraint 7); 1,350,455 were located outside low complexity regions (e.g., tandem repeats) (Constraint 8); and 1,302,588 had no identical or nearly identical paralogs (Constraint 9). After restricting sites to Chromosomes 1-22 (Constraint 10), 1,279,295 sites remained.

Second, a series of five constraints based on clinical criteria were applied as prerequisites for association with disease. The number of sites fell to 455,413 when restricted to those measured in at least 25 of the HPG patients (Constraint 11). Next, 40,860 remained after restriction to those with alternate allele frequencies in HPG patients that differed more than 0.05 from 1000G Phase 1 frequencies (Constraint 12a). Of these, 8,336 also exceeded by more than 0.05 the frequencies measured in the European subset of 1000G Phase 3 (Constraint 12b); 7,985 exceeded by more than 0.05 the frequencies measured in the European subset of the Exome Sequencing Project (Constraint 12c). To minimize false positives, sites were further restricted to those with frequencies that exceeded by more than 0.10 the frequencies in any of the comparison databases, leaving 2,235 sites (Constraint 13).

Third, two gene-based criteria further restricted sites. 1,408 sites remained when sites between genes were removed because intergenic sites found in sequencing are off target (Constraint 14). Of these, 933 were in genes detected as expressed in ocular tissues in associated laboratory studies (Constraint 15).

Fourth, we applied three statistical filters. Odds ratio and confidence interval were calculated for each site based on number of patients in whom the site was measured, the number of alternate alleles observed, and the number of measured and alternate alleles in the 1000G Phase 3 database. 506 sites had a 95% odds ratio confidence interval lower bound above 1.0 (Constraint 16). Data were fit with least squares linear regression to identify all sites above the fitted line, leaving 199 sites (Constraint 17). A 2×2 Fishers Exact Test was applied to obtain p-values. Sites were grouped by the number of patients, N, in which they had been measured, and a significance threshold was calculated for each measurement group using the Bonferroni formula (0.05/N) to correct for multiple testing. A final 160 sites remained significant after correction for multiple testing (Constraint 18). A total of 140 genes contained the 160 sites. See, Table 2. Variant sites evaluated in Constraints 13-18 are shown in Table 2.

For sites remaining after filtering, 53 (33%) each occurred in 25 to 49 of the HPG patients, and 107 (67%) each occurred in at least 50 of the HPG patients. Due to fluctuation in DNA capture efficiency, sites located in introns farther from exon splice sites tended to have smaller numbers of observations.

The 160 SNP sites are found in 140 genes. While 12 genes contained 2 SNP sites and 4 genes contained 3 SNP sites, 124 of the 140 genes contained a single SNP site. The genes are distributed across the genome. The nomenclature and sequence identification of these genes and other biomarkers described herein are known in the art and incorporated herein by reference (e.g., HUGO Gene Nomenclature Committee, National Center for Biotechnology Information, NCBI; GenBank accession numbers). See, Tables 3 and 4. (Table 3, Abbreviations: SNP, single nucleotide polymorphism; bp, basepairs; utr, UTR, untranslated regions. Table 4, Abbreviations: Gene identifiers obtained from the Human Genome Nomenclature Committee from genenames.org, with data downloaded from ftp://ftp.ebi.ac.uk/pub/databases/genenames/new/tsv/hgnc_complete_set.txt. [37] HPG, High Pressure Primary Open Angle Glaucoma; CHR, Chromosome; REF, hg19 Reference base; ALT, alternate base observed in HPG patients; dbSNP, NCBI identifier; missense, site position in codon, amino acid changes in sequence translated from mRNA on replacement of REF base with ALT base; synonym, site position in codon, no change in amino acid sequence translated from mRNA on replacement of REF base with ALT base; utr-3p, transcribed but untranslated region of mRNA in final (3′) exon; utr-5p, UTR in first (5p) exon; utr-NC, UTR in internal exon; SS DIST, distance to splicesite; OR, Odds ratio; Conf. Int., Confidence interval; pValue, probability that HPG and KG allele distributions are not different.)

These constraints reduced the number of SNP sites that are potentially more important in identifying genes that cause HPG from over 4 million sites to 160 sites in 140 genes. During filtering many SNP sites were set aside for further analysis.

Inhibitory nucleic acids or small inhibitory nucleic acids (siNAs) can be used in therapy treatments in combination with measurement of expression levels. For example, Tables 5 and 6 list microRNA differentially expressed in glaucomatous optic nerve (GON) versus normal optic nerve (ON or NON). microRNA underexpressed in GON can be neuroprotective when administered to a glaucoma patient. Targeting microRNA overexpressed in NON, e.g., with an inhibitory nucleic acid, can be neuroprotective to a glaucoma patient. Conversely, microRNA underexpressed in GON can be pathological and thus targeted, e.g., with an inhibitory nucleic acid, in a glaucoma patient; microRNA overexpressed in NON can be neuroprotective when administered to a glaucoma patient. (Table 5, Abbreviations: RT, RT n, retina; ON,ON n, optic nerve; GON, ON g, glaucomatous optic nerve; A>>B, “A significantly higher than B”. Table 6, Abbreviations: RT, RT n, retina; ON, NON, ON n, optic nerve; GON, ON g, glaucomatous optic nerve; MAX, maximum; A>>B, level in A significantly higher than B. microRNA names, miRbase [38] and Ambros, et al., 2002 [39];

TABLE 3
Properties of 140 genes and 160 SNP sites.
Gene properties

| | | |
|---|---|---|
| a. | 124 | w/1 SNP site |
| | 12 | w/2 SNP sites |
| | 4 | w/3 SNP sites |

SNP site properties

| | | |
|---|---|---|
| b. | 23 | Codon |
| | 118 | Intron |
| | 13 | utr-3p |
| | 4 | utr-5p |
| | 2 | utr-NC |
| c. | 12 | Missense |
| | 11 | synonymous |
| d. | 84 | intron, within 500 bp of splice site |
| | 34 | intron, >500 bp from splice site |

SNP site distance distributions

| | | |
|---|---|---|
| e. | 140 | 1st SNP site in gene |
| | 1 | SNP site adjacent to a 1^{st} site in gene |
| | 3 | SNP sites 2-3 bp of 1^{st} site in gene |
| | 9 | SNP sites 4-55 bp of 1^{st} site in gene |
| | 2 | SNP sites 150-250 bp of 1^{st} site in gene |
| f. | 24 | SNP sites within 100,000 bp of prior site |

Gene annotations, 85 genes (49 in multiple categories)

| | | |
|---|---|---|
| g. | 51 | Cell cycle, apoptosis, proliferation |
| | 33 | Neural-related |
| | 30 | Adhesion |
| | 28 | Immune-related |
| | 19 | Transcription factor or RNA binding |
| | 14 | Mitochondrial |
| | 11 | Ocular |
| h. | 5 | Prior glaucoma-related |
| i. | 1 | Prior glaucoma-related & neural & immune |
| | 1 | Prior glaucoma-related & retinal |

a. SNPs per gene, b. location in gene, c. codon effect, d. distance to splicesite, e. proximal SNPs within genes, f. proximal SNPs in adjacent genes, g. genes with functions relevant to glaucoma, h. prior glaucoma related genes, i. glaucoma related and relevant functions.

TABLE 4
160 SNP sites identifying 140 genes as risk variants for high pressure glaucoma (HPG)

| | | | | | | | | | | | | | | | |
|---|---|---|---|---|---|---|---|---|---|---|---|---|---|---|---|
| CHR | POSITION | REF | ALT | dbSNP | GENE | #Paralogs (if >1) | #HPG SNPs (if >1) | LOCATION | EFFECT | SS DIST | KG FQ | HPG FQ | OR | OR Conf. Int. | p Value |

4a. 134 of 140 Genes: strongest SNP site

| | | | | | | | | | | | | | | |
|---|---|---|---|---|---|---|---|---|---|---|---|---|---|---|
| 1 | 145,015,877 | G | T | rs77741369 | PDE4DIP | | codon | missense | 18 | 0.192 | 0.370 | 2.45 | 1.84-3.25 | 1.30E−09 |
| 3 | 195,506,914 | G | A | rs186560307 | MUC4 | 2 | codon | missense | 79 | 0.154 | 0.397 | 3.60 | 2.58-5.01 | 1.70E−13 |
| 11 | 22,271,870 | A | T | rs7481951 | ANO5 | | codon | missense | 48 | 0.349 | 0.576 | 2.54 | 1.92-3.35 | 3.90E−11 |
| 11 | 117,789,345 | G | C | rs61900347 | TMPRSS13 | 2 | codon | missense | 209 | 0.100 | 0.443 | 7.17 | 5.25-9.79 | 1.60E−35 |
| 14 | 105,415,748 | G | A | rs118171013 | AHNAK2 | | codon | missense | 5,389 | 0.301 | 0.544 | 2.75 | 2.09-3.60 | 2.10E−13 |
| 15 | 74,336,633 | T | C | rs5742915 | PML | | codon | missense | 67 | 0.226 | 0.460 | 2.90 | 2.19-3.83 | 1.40E−13 |
| 19 | 7,935,879 | G | A | rs12984448 | FLJ22184 | | codon | missense | 1,279 | 0.071 | 0.263 | 4.55 | 2.78-7.44 | 2.40E−08 |
| 1 | 87,045,902 | A | T | rs1932809 | CLCA4 | | codon | synonym | 278 | 0.231 | 0.677 | 6.94 | 5.14-9.35 | 6.10E−40 |
| 1 | 181,759,614 | A | C | rs35611740 | CACNA1E | | codon | synonym | 34 | 0.013 | 0.198 | 18.95 | 10.00-35.89 | 1.20E−23 |
| 2 | 216,973,904 | C | A | rs1647764 | XRCC5 | | codon | synonym | 91 | 0.054 | 0.205 | 4.48 | 2.62-7.63 | 3.90E−07 |
| 3 | 122,642,590 | G | A | rs2276778 | SEMA5B | | codon | synonym | 10 | 0.432 | 0.640 | 2.31 | 1.75-3.04 | 1.90E−09 |
| 4 | 140,810,700 | C | T | rs11729794 | MAML3 | | codon | synonym | 190 | 0.259 | 0.482 | 2.63 | 2.00-3.45 | 5.60E−12 |
| 7 | 5,352,659 | G | T | rs138591330 | TNRC18 | | codon | synonym | 526 | 0.348 | 0.680 | 3.99 | 2.39-6.65 | 4.10E−08 |
| 9 | 79,318,378 | G | A | rs13290609 | PRUNE2 | 3 | codon | synonym | 126 | 0.328 | 0.700 | 4.79 | 3.55-6.44 | 5.00E−27 |
| 16 | 70,726,795 | C | A | rs2278983 | VAC14 | | codon | synonym | 72 | 0.283 | 0.471 | 2.26 | 1.71-2.98 | 1.00E−08 |
| 17 | 5,085,389 | C | T | rs148322165 | ZNF594 | | codon | synonym | 2,183 | 0.021 | 0.260 | 16.56 | 9.17-29.89 | 1.30E−19 |
| 20 | 56,137,834 | A | G | rs1062601 | PCK1 | | codon | synonym | 83 | 0.322 | 0.512 | 2.19 | 1.66-2.87 | 1.60E−08 |
| 5 | 138,665,756 | G | A | rs7305 | MATR3 | | utr-3p | | 723 | 0.410 | 0.690 | 3.12 | 1.97-4.92 | 4.70E−07 |
| 6 | 33,989,811 | C | G | rs186466095 | GRM4 | | utr-3p | | 837 | 0.028 | 0.196 | 8.25 | 4.47-15.22 | 9.20E−10 |
| 12 | 96,672,743 | G | C | rs3087520 | CDK17 | | utr-3p | | 1,315 | 0.145 | 0.322 | 2.81 | 1.58-4.98 | 1.10E−03 |
| 14 | 90,873,412 | C | G | rs3179089 | CALM1 | | utr-3p | | 2,380 | 0.337 | 0.589 | 2.82 | 1.63-4.87 | 2.00E−04 |
| 15 | 28,356,859 | C | T | rs1129038 | HERC2 | | utr-3p | | 323 | 0.293 | 0.699 | 5.63 | 4.17-7.59 | 2.30E−32 |
| 15 | 59,429,160 | G | A | rs3191402 | MYO1E | | utr-3p | | 496 | 0.193 | 0.386 | 2.59 | 1.72-3.89 | 1.00E−05 |
| 16 | 29,677,845 | T | A | rs11574560 | SPN | | utr-3p | | 2,830 | 0.248 | 0.520 | 3.29 | 2.16-4.99 | 4.90E−08 |
| 16 | 85,009,552 | T | G | rs9934780 | ZDHHC7 | | utr-3p | | 574 | 0.310 | 0.588 | 3.18 | 1.79-5.64 | 8.30E−05 |
| 17 | 75,212,335 | C | A | rs147840397 | SEC14L1 | 3 | utr-3p | | 294 | 0.453 | 0.738 | 3.34 | 2.21-5.03 | 1.60E−09 |
| 19 | 44,501,929 | G | C | rs11881151 | ZNF155 | | utr-3p | | 1,685 | 0.489 | 0.741 | 3.01 | 1.33-6.79 | 5.80E−03 |
| 2 | 46,746,675 | G | A | rs13023346 | ATP6V1E2 | | utr-5p | | 592 | 0.361 | 0.671 | 3.38 | 1.66-6.87 | 5.00E−04 |
| 4 | 48,655,845 | C | A | rs73815308 | FRYL | | utr-5p | | 115 | 0.043 | 0.225 | 6.71 | 3.84-11.71 | 1.10E−09 |
| 9 | 118,342 | T | C | rs2492221 | FOXD4 | | utr-5p | | 3,232 | 0.156 | 0.325 | 2.59 | 1.71-3.91 | 1.40E−05 |
| 17 | 35,306,312 | G | C | rs2306658 | AATF | | utr-5p | | 205 | 0.317 | 0.535 | 2.48 | 1.67-3.66 | 5.80E−06 |
| 2 | 239,141,724 | G | A | rs11695827 | LOC643387 | | utr-NC | | 1,750 | 0.205 | 0.386 | 2.44 | 1.60-3.71 | 6.50E−05 |
| 1 | 27,995,565 | C | T | rs71514291 | IFI6 | | intron | | 165 | 0.489 | 0.978 | 43.69 | 19.26-99.09 | 1.00E−57 |
| 1 | 148,741,823 | T | G | rs187644417 | NBPF16 | | intron | | 29 | 0.360 | 0.820 | 7.69 | 4.05-14.59 | 1.10E−12 |
| 1 | 161,022,639 | C | G | rs3813610 | ARHGAP30 | | intron | | 50 | 0.430 | 0.648 | 2.41 | 1.72-3.36 | 1.70E−07 |
| 1 | 177,043,591 | C | G | rs11587524 | ASTN1 | | intron | | 13,188 | 0.330 | 0.590 | 2.93 | 1.58-5.42 | 5.00E−04 |
| 1 | 205,689,807 | T | G | rs823094 | NUCKS1 | | intron | | 24 | 0.247 | 0.438 | 2.38 | 1.78-3.17 | 5.30E−09 |
| 1 | 213,173,457 | C | T | rs3738806 | ANGEL2 | | intron | | 209 | 0.447 | 0.695 | 2.88 | 1.84-4.49 | 1.60E−06 |
| 1 | 236,902,364 | C | T | rs4659711 | ACTN2 | | intron | | 236 | 0.402 | 0.649 | 2.75 | 1.57-4.80 | 2.00E−04 |
| 1 | 248,018,426 | C | T | rs7513527 | TRIM58 | | intron | | 2,543 | 0.194 | 0.422 | 3.03 | 1.64-5.58 | 9.00E−04 |
| 2 | 26,606,347 | T | A | rs13030294 | EPT1 | 2 | intron | | 130 | 0.302 | 0.589 | 3.24 | 2.08-5.04 | 1.90E−07 |
| 2 | 86,398,074 | A | G | rs2288115 | IMMT | | intron | | 105 | 0.283 | 0.510 | 2.64 | 1.91-3.63 | 4.30E−09 |
| 2 | 97,857,179 | G | C | rs13032546 | ANKRD36 | | intron | | 259 | 0.434 | 0.793 | 4.96 | 3.49-7.04 | 1.50E−22 |
| 2 | 98,306,608 | C | A | rs12997695 | LOC728537 | | intron | | 109 | 0.377 | 0.701 | 3.76 | 2.31-6.11 | 3.00E−08 |
| 2 | 136,411,879 | A | G | rs76622824 | R3HDM1 | | intron | | 2,275 | 0.310 | 0.590 | 3.02 | 1.49-6.10 | 2.00E−03 |
| 2 | 234,070,190 | G | A | rs28609111 | INPP5D | | intron | | 178 | 0.243 | 0.463 | 2.68 | 1.82-3.92 | 7.90E−07 |
| 3 | 56,591,298 | T | A | rs73079892 | CCDC66 | 2 | intron | | 15 | 0.201 | 0.474 | 3.56 | 2.68-4.71 | 1.80E−18 |
| 3 | 98,537,835 | T | C | rs9823430 | DCBLD2 | | intron | | 209 | 0.342 | 0.534 | 2.21 | 1.19-4.08 | 1.34E−02 |
| 3 | 100,594,332 | A | C | rs201168844 | ABI3BP | | intron | | 18 | 0.038 | 0.311 | 11.59 | 7.56-17.74 | 8.30E−31 |
| 3 | 140,282,084 | C | G | rs4683509 | CLSTN2 | | intron | | 37 | 0.474 | 0.799 | 4.36 | 3.11-6.10 | 1.30E−20 |
| 3 | 182,810,144 | T | G | rs3732604 | MCCC1 | | intron | | 51 | 0.424 | 0.676 | 2.80 | 2.05-3.81 | 2.60E−11 |
| 3 | 186,302,392 | G | T | rs78484175 | DNAJB11 | | intron | | 12 | 0.381 | 0.619 | 2.64 | 1.83-3.79 | 9.20E−08 |
| 4 | 25,759,110 | T | C | rs201172550 | SEL1L3 | | intron | | 44 | 0.188 | 0.500 | 4.46 | 2.59-7.67 | 1.40E−07 |
| 4 | 62,641,105 | C | A | rs28735546 | LPHN3 | | intron | | 38,420 | 0.378 | 0.796 | 5.75 | 2.30-14.37 | 6.10E−05 |
| 4 | 124,322,633 | C | A | rs300573 | SPRY1 | | intron | | 57 | 0.433 | 0.655 | 2.49 | 1.74-3.56 | 3.10E−07 |
| 5 | 7,534,899 | C | G | rs1553245 | ADCY2 | | intron | | 13,885 | 0.428 | 0.730 | 3.63 | 1.51-8.71 | 2.40E−03 |
| 5 | 9,157,202 | G | T | rs988592 | SEMA5A | | intron | | 2,393 | 0.470 | 0.758 | 3.25 | 1.44-7.33 | 3.10E−03 |
| 5 | 82,871,546 | T | C | rs309581 | VCAN | | intron | | 3,165 | 0.373 | 0.666 | 3.35 | 1.48-7.53 | 3.90E−03 |
| 5 | 153,029,856 | T | A | rs112508290 | GRIA1 | | intron | | 32 | 0.024 | 0.176 | 8.80 | 5.20-14.86 | 2.40E−16 |
| 5 | 169,701,546 | G | C | rs4867953 | LCP2 | | intron | | 202 | 0.331 | 0.528 | 2.21 | 1.42-3.42 | 4.00E−04 |
| 5 | 177,696,914 | C | T | rs2973757 | COL23A1 | | intron | | 422 | 0.426 | 0.787 | 5.00 | 2.15-11.62 | 4.60E−05 |
| 5 | 179,980,215 | A | C | rs11738044 | CNOT6 | | intron | | 169 | 0.361 | 0.662 | 3.28 | 1.69-6.36 | 3.00E−04 |
| 6 | 29,912,510 | T | G | rs114688017 | HLA-A | | intron | | 115 | 0.329 | 0.535 | 2.30 | 1.51-3.49 | 9.10E−05 |
| 6 | 30,892,377 | G | C | rs115696940 | VARS2 | | intron | | 38 | 0.244 | 0.430 | 2.30 | 1.53-3.45 | 1.00E−04 |
| 6 | 32,548,068 | T | A | rs4498414 | HLA-DRB1 | 2 | intron | | 19 | 0.161 | 0.376 | 3.07 | 2.04-4.60 | 1.80E−07 |
| 6 | 69,666,195 | G | A | rs28734080 | BAI3 | | intron | | 113 | 0.449 | 0.701 | 2.75 | 1.50-5.01 | 8.00E−04 |
| 6 | 155,152,311 | C | G | rs911191 | SCAF8 | | intron | | 35 | 0.322 | 0.544 | 2.46 | 1.58-3.80 | 5.90E−05 |
| 7 | 151,519,128 | A | C | rs11770376 | PRKAG2 | | intron | | 7,627 | 0.373 | 0.628 | 2.68 | 1.38-5.17 | 3.70E−03 |
| 8 | 17,871,619 | A | C | rs28551649 | PCM1 | | intron | | 72 | 0.362 | 0.731 | 4.79 | 2.37-9.67 | 3.50E−06 |
| 8 | 25,767,802 | T | C | rs11783374 | EBF2 | | intron | | 1,729 | 0.485 | 0.700 | 2.65 | 1.25-5.57 | 9.20E−03 |
| 8 | 71,581,006 | T | G | rs268645 | LACTB2 | | intron | | 226 | 0.477 | 0.838 | 5.11 | 2.09-12.44 | 7.00E−05 |
| 8 | 118,183,658 | C | A | rs2466298 | SLC30A8 | | intron | | 249 | 0.481 | 0.790 | 4.07 | 1.93-8.57 | 6.50E−05 |
| 8 | 121,583,283 | A | G | rs4455882 | SNTB1 | | intron | | 322 | 0.388 | 0.750 | 4.72 | 1.98-11.20 | 2.00E−04 |
| 9 | 41,954,734 | G | A | rs28714309 | MGC21881 | 2 | intron | | 13 | 0.042 | 0.292 | 9.31 | 5.48-15.81 | 3.70E−14 |
| 9 | 112,427,544 | C | T | rs7038227 | PALM2 | | intron | | 24,396 | 0.085 | 0.285 | 4.29 | 2.11-8.68 | 2.00E−04 |
| 9 | 125,748,867 | G | C | rs676805 | RABGAP1 | | intron | | 167 | 0.498 | 0.915 | 9.67 | 3.81-24.48 | 9.80E−10 |
| 9 | 132,576,665 | C | A | rs13294595 | TOR1A | | intron | | 162 | 0.286 | 0.700 | 6.23 | 2.95-13.13 | 3.30E−07 |
| 9 | 132,632,178 | C | T | rs62583580 | USP20 | | intron | | 53 | 0.081 | 0.297 | 4.64 | 2.71-7.92 | 2.40E−07 |
| 9 | 136,599,534 | T | C | rs10993781 | SARDH | | intron | | 189 | 0.409 | 0.757 | 4.51 | 2.01-10.09 | 9.60E−05 |
| 10 | 285,113 | C | G | rs17221239 | ZMYND11 | | intron | | 263 | 0.205 | 0.465 | 3.15 | 1.49-6.65 | 4.10E−03 |
| 10 | 27,087,849 | A | C | rs10829066 | ABI1 | | intron | | 21,677 | 0.142 | 0.481 | 5.62 | 2.58-12.20 | 3.70E−05 |
| 10 | 69,651,125 | A | G | rs7896005 | SIRT1 | | intron | | 33 | 0.374 | 0.676 | 3.44 | 2.40-4.92 | 3.50E−12 |
| 10 | 73,046,177 | G | T | rs10823717 | UNC5B | | intron | | 267 | 0.316 | 0.535 | 2.49 | 1.44-4.27 | 1.10E−03 |
| 10 | 88,006,977 | A | G | rs61856566 | GRID1 | | intron | | 40,570 | 0.092 | 0.263 | 3.56 | 1.67-7.55 | 2.20E−03 |
| 10 | 99,660,299 | A | G | rs642752 | CRTAC1 | | intron | | 959 | 0.230 | 0.416 | 2.39 | 1.51-3.77 | 2.00E−04 |
| 10 | 104,708,312 | A | G | rs12783467 | CNNM2 | | intron | | 21,211 | 0.218 | 0.427 | 2.56 | 1.41-4.63 | 2.40E−03 |
| 11 | 1,971,925 | G | T | rs217201 | MRPL23 | 2 | intron | | 202 | 0.270 | 0.642 | 4.85 | 2.54-9.25 | 1.40E−06 |
| 11 | 46,924,665 | C | G | rs12806687 | LRP4 | | intron | | 183 | 0.433 | 0.658 | 2.52 | 1.55-4.07 | 1.00E−04 |
| 11 | 71,153,459 | C | A | rs11603330 | DHCR7 | | intron | | 58 | 0.462 | 0.708 | 2.79 | 2.08-3.72 | 9.20E−13 |
| 11 | 113,294,998 | T | A | rs12808482 | DRD2 | | intron | | 85 | 0.333 | 0.637 | 3.44 | 2.32-5.09 | 3.70E−10 |
| 11 | 130,338,012 | C | T | rs11600748 | ADAMTS15 | | intron | | 1,143 | 0.414 | 0.656 | 2.63 | 1.63-4.23 | 5.40E−05 |
| 12 | 7,655,278 | T | A | rs11836971 | CD163 | | intron | | 116 | 0.498 | 0.764 | 3.26 | 1.96-5.39 | 1.20E−06 |
| 12 | 9,463,968 | G | A | rs12366847 | LOC642846 | | intron | | 80 | 0.304 | 0.557 | 2.87 | 2.13-3.84 | 1.40E−12 |
| 12 | 10,275,684 | T | G | rs11053608 | CLEC7A | | intron | | 158 | 0.337 | 0.609 | 3.07 | 1.95-4.81 | 1.00E−06 |
| 12 | 31,132,243 | T | A | rs720480 | TSPAN11 | | intron | | 256 | 0.247 | 0.444 | 2.42 | 1.83-3.19 | 6.10E−10 |
| 12 | 65,633,886 | G | C | rs201470777 | LEMD3 | | intron | | 28 | 0.004 | 0.273 | 94.90 | 34.02-264.70 | 2.30E−43 |
| 12 | 81,231,660 | T | C | rs7309585 | LIN7A | 2 | intron | | 4,248 | 0.405 | 0.724 | 3.85 | 1.68-8.77 | 8.00E−04 |
| 12 | 81,661,039 | G | A | rs12826016 | PPFIA2 | | intron | | 238 | 0.227 | 0.412 | 2.39 | 1.55-3.67 | 1.00E−04 |
| 12 | 104,651,594 | A | G | rs7297584 | TXNRD1 | | intron | | 201 | 0.256 | 0.547 | 3.51 | 1.88-6.54 | 1.00E−04 |
| 12 | 133,352,788 | C | G | rs184432110 | GOLGA3 | | intron | | 430 | 0.043 | 0.378 | 12.66 | 6.26-25.58 | 5.90E−10 |
| 13 | 111,145,427 | T | C | rs9555711 | COL4A2 | | intron | | 129 | 0.133 | 0.339 | 3.33 | 2.28-4.84 | 2.00E−09 |
| 13 | 113,616,369 | G | T | rs2185001 | MCF2L | | intron | | 6,660 | 0.281 | 0.882 | 19.17 | 6.69-54.90 | 1.00E−12 |
| 14 | 21,895,605 | A | G | rs12885579 | CHD8 | | intron | | 153 | 0.352 | 0.796 | 7.13 | 5.02-10.10 | 2.40E−34 |
| 14 | 30,046,816 | G | T | rs8010772 | PRKD1 | | intron | | 152 | 0.388 | 0.596 | 2.33 | 1.38-3.93 | 1.80E−03 |
| 14 | 62,014,289 | T | A | rs2184633 | PRKCH | | intron | | 170 | 0.459 | 0.764 | 3.82 | 2.15-6.78 | 1.00E−06 |
| 14 | 68,805,451 | C | G | rs2013413 | RAD51B | | intron | | 46,752 | 0.457 | 0.671 | 2.37 | 1.55-3.62 | 5.30E−05 |
| 14 | 73,256,150 | T | C | rs4903059 | DPF3 | | intron | | 17,547 | 0.076 | 0.397 | 7.62 | 3.97-14.59 | 3.30E−08 |
| 14 | 93,049,739 | C | G | rs12589396 | RIN3 | | intron | | 5,915 | 0.420 | 0.765 | 4.14 | 1.84-9.30 | 3.00E−04 |
| 15 | 66,427,278 | A | C | rs4776743 | MEGF11 | | intron | | 6,527 | 0.321 | 0.562 | 2.72 | 1.33-5.53 | 6.50E−03 |
| 16 | 2,810,623 | T | G | rs2285879 | SRRM2 | | intron | | 121 | 0.431 | 0.687 | 2.90 | 1.55-5.40 | 5.00E−04 |
| 16 | 5,690,409 | A | G | rs55804704 | RBFOX1 | 2 | intron | | 41,445 | 0.415 | 0.718 | 3.43 | 1.89-6.21 | 2.00E−05 |
| 16 | 20,811,092 | G | T | rs4783504 | ERI2 | | intron | | 185 | 0.342 | 0.611 | 3.02 | 1.52-5.97 | 1.20E−03 |
| 16 | 82,660,946 | T | C | rs62040565 | CDH13 | | intron | | 202 | 0.229 | 0.432 | 2.55 | 1.30-4.96 | 8.90E−03 |
| 16 | 88,716,394 | C | A | rs33997949 | CYBA | | intron | | 968 | 0.356 | 0.619 | 2.93 | 1.55-5.53 | 8.00E−04 |
| 16 | 88,774,921 | C | T | rs7404845 | CTU2 | | intron | | 1,301 | 0.195 | 0.469 | 3.43 | 1.69-6.92 | 1.30E−03 |
| 17 | 4,795,307 | C | T | rs2586532 | MINK1 | | intron | | 135 | 0.477 | 0.782 | 3.95 | 2.63-5.92 | 6.30E−13 |
| 17 | 4,859,123 | T | C | rs111645512 | ENO3 | 3 | intron | | 112 | 0.328 | 0.619 | 3.34 | 2.26-4.93 | 7.70E−10 |
| 17 | 5,074,623 | A | G | rs11078550 | USP6 | | intron | | 257 | 0.388 | 0.660 | 3.15 | 1.91-5.17 | 4.10E−06 |
| 17 | 10,021,329 | T | A | rs11653469 | GAS7 | | intron | | 58,321 | 0.304 | 0.564 | 2.78 | 1.35-5.71 | 5.50E−03 |
| 17 | 44,326,619 | A | G | rs149459294 | LRRC37A | 3 | intron | | 1,092 | 0.264 | 0.512 | 2.92 | 1.53-5.55 | 1.40E−03 |
| 17 | 73,729,445 | C | A | rs12600897 | ITGB4 | | intron | | 124 | 0.418 | 0.765 | 4.18 | 1.85-9.39 | 2.00E−04 |
| 19 | 2,567,889 | C | T | rs34867917 | GNG7 | | intron | | 12,701 | 0.183 | 0.358 | 2.51 | 1.28-4.92 | 1.06E−02 |
| 19 | 3,281,944 | G | T | rs34139199 | CELF5 | | intron | | 178 | 0.420 | 0.733 | 3.69 | 2.41-5.64 | 1.70E−10 |
| 19 | 10,421,385 | G | C | rs281418 | FDX1L | | intron | | 65 | 0.227 | 0.408 | 2.35 | 1.57-3.50 | 4.90E−05 |
| 19 | 16,320,075 | C | G | rs2290802 | AP1M1 | | intron | | 85 | 0.488 | 0.741 | 2.97 | 2.02-4.34 | 3.90E−09 |
| 19 | 33,695,241 | G | T | rs8100642 | LRP3 | | intron | | 301 | 0.269 | 0.620 | 4.29 | 2.52-7.28 | 5.40E−08 |
| 19 | 38,269,986 | A | C | rs17306900 | ZNF573 | | intron | | 196 | 0.212 | 0.472 | 3.20 | 1.83-5.57 | 7.10E−05 |
| 19 | 53,122,155 | A | C | rs35672405 | ZNF83 | 2 | intron | | 32 | 0.117 | 0.337 | 3.84 | 2.67-5.52 | 3.40E−12 |
| 20 | 59,858,617 | T | G | rs2427025 | CDH4 | | intron | | 28,622 | 0.170 | 0.352 | 2.52 | 1.32-4.80 | 7.40E−03 |
| 21 | 30,252,088 | T | A | rs2205449 | N6AMT1 | | intron | | 102 | 0.378 | 0.750 | 5.28 | 2.56-10.86 | 9.70E−07 |
| 22 | 36,691,969 | G | A | rs875726 | MYH9 | | intron | | 204 | 0.449 | 0.733 | 3.25 | 1.83-5.75 | 2.00E−05 |
| 22 | 36,891,858 | C | A | rs5756219 | FOXRED2 | | intron | | 154 | 0.303 | 0.601 | 3.43 | 2.43-4.82 | 1.10E−12 |
| 22 | 40,015,493 | C | T | rs12628643 | CACNA1I | | intron | | 79 | 0.489 | 0.718 | 2.54 | 1.40-4.60 | 2.00E−03 |
| 22 | 45,595,278 | G | C | rs2235159 | KIAA0930 | 2 | intron | | 474 | 0.443 | 0.765 | 4.12 | 2.73-6.20 | 3.20E−13 |

4b. 6 of 140 genes with ≧90% identical paralog, strongest SNP site (5 with 1 SNP)

| | | | | | | | | | | | | | | | |
|---|---|---|---|---|---|---|---|---|---|---|---|---|---|---|---|
| 2 | 98,166,215 | C | G | rs6750205 | ANKRD36B | 2 | | intron | | 136 | 0.470 | 0.691 | 2.50 | 1.88-3.31 | 6.20E−11 |
| 9 | 15,883 | A | G | rs141156662 | WASH1 | 7 | | intron | | 24 | 0.192 | 0.500 | 4.27 | 2.86-6.35 | 3.60E−12 |
| 16 | 18,531,692 | T | C | rs137862509 | NOMO2 | 2 | | intron | | 225 | 0.225 | 0.543 | 4.10 | 2.25-7.46 | 7.50E−06 |
| 18 | 14,529,901 | A | G | rs62081684 | POTEC | 2 | | intron | | 579 | 0.303 | 0.580 | 3.45 | 1.53-7.76 | 3.30E−03 |
| 19 | 54,724,457 | T | C | rs180678650 | LILRB3 | 1 | 2 | codon | missense | 60 | 0.168 | 0.673 | 10.21 | 5.39-19.32 | 2.20E−13 |
| 19 | 54,778,909 | A | G | rs117474097 | LILRB2 | 2 | | intron | | 224 | 0.029 | 0.263 | 11.96 | 7.50-19.06 | 2.00E−27 |

4c. 2nd SNP site for 16 of 140 genes

| | | | | | | | | | | | | | | | |
|---|---|---|---|---|---|---|---|---|---|---|---|---|---|---|---|
| 2 | 26,618,543 | G | A | rs7092 | EPT1 | | 2 | utr-3p | | 6,470 | 0.282 | 0.564 | 3.31 | 2.18-5.02 | 2.40E−08 |
| 3 | 56,599,208 | G | C | rs55831745 | CCDC66 | | 2 | intron | | 1,053 | 0.283 | 0.582 | 3.54 | 2.28-5.48 | 1.80E−08 |
| 3 | 195,505,907 | T | G | rs138720131 | MUC4 | | 2 | codon | missense | 161 | 0.175 | 0.344 | 2.46 | 1.74-3.46 | 7.50E−07 |
| 6 | 32,548,122 | C | G | rs4448132 | HLA-DRB1 | | 2 | intron | | 73 | 0.130 | 0.338 | 3.35 | 2.12-5.28 | 8.60E−07 |
| 9 | 41,954,776 | C | G | rs28706047 | MGC21881 | | 2 | utr-NC | | 28 | 0.322 | 0.573 | 2.78 | 1.86-4.15 | 7.00E−07 |
| 9 | 79,318,381 | A | T | rs113471142 | PRUNE2 | | 3 | codon | synonym | 129 | 0.237 | 0.593 | 4.66 | 3.41-6.36 | 1.60E−22 |
| 11 | 1,971,918 | A | C | rs217200 | MRPL23 | | 2 | intron | | 209 | 0.178 | 0.530 | 4.89 | 2.42-9.86 | 1.70E−05 |
| 11 | 117,789,327 | T | C | rs61900346 | TMPRSS13 | | 2 | codon | missense | 204 | 0.187 | 0.428 | 3.23 | 2.42-4.29 | 2.20E−15 |
| 12 | 81,231,631 | A | G | rs12318213 | LIN7A | | 2 | intron | | 4,277 | 0.309 | 0.523 | 2.34 | 1.26-4.31 | 7.00E−03 |
| 16 | 7,008,029 | C | T | rs12917775 | RBFOX1 | | 2 | intron | | 94,027 | 0.185 | 0.440 | 3.45 | 1.54-7.72 | 3.40E−03 |
| 17 | 4,859,156 | C | T | rs146545678 | ENO3 | | 3 | intron | | 79 | 0.156 | 0.378 | 3.25 | 2.26-4.66 | 1.10E−09 |
| 17 | 44,340,253 | A | G | rs113507264 | LRRC37A | | 3 | intron | | 12,532 | 0.249 | 0.500 | 3.23 | 1.53-6.78 | 2.10E−03 |
| 17 | 75,212,491 | C | A | rs1130549 | SEC14L1 | | 3 | utr-3p | | 138 | 0.177 | 0.349 | 2.48 | 1.77-3.46 | 3.20E−07 |
| 19 | 53,122,146 | A | C | rs35489438 | ZNF83 | | 2 | intron | | 41 | 0.112 | 0.304 | 3.48 | 2.40-5.02 | 1.90E−10 |
| 19 | 54,724,458 | A | G | rs185399462 | LILRB3 | 1 | 2 | codon | missense | 61 | 0.284 | 0.684 | 5.20 | 2.76-9.77 | 1.40E−07 |
| 22 | 45,595,265 | C | G | rs78963667 | KIAA0930 | | 2 | intron | | 487 | 0.130 | 0.318 | 3.09 | 2.08-4.56 | 7.20E−08 |

4d. 3rd SNP site for 4 of 140 genes

| | | | | | | | | | | | | | | |
|---|---|---|---|---|---|---|---|---|---|---|---|---|---|---|
| 9 | 79,318,384 | C | A | rs199893827 | PRUNE2 | 3 | codon | missense | 132 | 0.141 | 0.334 | 3.02 | 2.06-4.42 | 5.90E−08 |
| 17 | 4,859,134 | C | T | rs117488294 | ENO3 | 3 | intron | | 101 | 0.248 | 0.500 | 3.07 | 2.05-4.58 | 6.10E−08 |
| 17 | 44,326,845 | A | G | rs118111151 | LRRC37A | 3 | intron | | 1,318 | 0.263 | 0.447 | 2.26 | 1.17-4.34 | 1.56E−02 |
| 17 | 75,212,489 | T | C | rs62079472 | SEC14L1 | 3 | utr-3p | | 140 | 0.166 | 0.339 | 2.56 | 1.80-3.62 | 3.90E−07 |

TABLE 5
43 microRNAs differentially regulated in glaucoma optic nerve (GON) vs normal optic nerve (ON) and
targeting HPG genes, with microRNA name and the mature arm with strongest differential expression.

| | microRNA | Stronger arm | GON level | ON level | RT level | log2 ON g/ON n | log2 ON n/RT n |
|---|---|---|---|---|---|---|---|
| Group 1 | hsa-miR-130a | hsa-miR-130a-3p | 46,075 | 21,770 | 14,388 | 1 | 1 |
| 8 up GON>>ON | hsa-miR-1246 | hsa-miR-1246 | 1,990 | 1,093 | 421 | 1 | 1 |
| ON >> RT | hsa-miR-214 | hsa-miR-214-3p | 3,655 | 2,018 | 32 | 1 | 6 |
| | hsa-miR-452 | hsa-miR-452-5p | 1,258 | 381 | 41 | 2 | 3 |
| | hsa-miR-224 | hsa-miR-224-5p | 741 | 306 | 49 | 1 | 3 |
| | hsa-miR-4448 | hsa-miR-4448 | 720 | 195 | 27 | 2 | 3 |
| | hsa-miR-483 | hsa-miR-483-5p | 1,365 | 353 | 1 | 2 | 7 |
| | hsa-miR-483 | hsa-miR-483-3p | 505 | 231 | 1 | 1 | 7 |
| Group 2 | hsa-miR-9 | hsa-miR-9-3p | 12,281 | 5,817 | 57,284 | 1 | −3 |
| 3 up GON >> ON | hsa-miR-767 | hsa-miR-767-5p | 290 | 83 | 890 | 2 | −3 |
| RT >> ON | hsa-miR-449a | hsa-miR-449a | 376 | 1 | 27 | 8 | −4 |
| Group 3 | hsa-miR-100 | hsa-miR-100-5p | 260,330 | 403,037 | 50,309 | −1 | 3 |
| 7 down GON | hsa-miR-219 | hsa-miR-219-5p | 92,592 | 226,290 | 454 | −1 | 9 |
| ON >> RT | hsa-miR-219 | hsa-miR-219-2-3p | 80,029 | 121,995 | 23 | −1 | 12 |
| | hsa-miR-99b | hsa-miR-99b-5p | 77,728 | 123,776 | 37,955 | −1 | 2 |
| | hsa-miR-139 | hsa-miR-139-5p | 1,067 | 2,434 | 1,077 | −1 | 1 |
| | hsa-miR-323b | hsa-miR-323b-3p | 466 | 1,772 | 913 | −2 | 1 |
| | hsa-miR-3613 | hsa-miR-3613-3p | 268 | 493 | 44 | −1 | 3 |
| Group 4 | hsa-miR-124 | hsa-miR-124-3p | 8,155 | 21,083 | 90,984 | −1 | −2 |
| 4 down GON << ON | hsa-miR-129 | hsa-miR-129-5p | 1,281 | 3,925 | 11,942 | −2 | −2 |
| RT >> ON | hsa-miR-211 | hsa-miR-211-5p | 279 | 1,283 | 20,755 | −2 | −4 |
| | hsa-miR-124 | hsa-miR-124-5p | 165 | 316 | 18,110 | −1 | −6 |
| Group 5 | hsa-miR-34b | hsa-miR-34b-3p | 0 | 125 | 0 | −7 | 7 |
| 16 off in GON | hsa-miR-3182 | hsa-miR-3182 | 0 | 106 | 0 | −7 | 7 |
| ON >> RT | hsa-miR-4640 | hsa-miR-4640-3p | 0 | 99 | 0 | −7 | 7 |
| | hsa-miR-2276 | hsa-miR-2276 | 0 | 74 | 0 | −6 | 6 |
| | hsa-miR-4423 | hsa-miR-4423-5p | 0 | 65 | 0 | −6 | 6 |
| | hsa-miR-2277 | hsa-miR-2277-3p | 0 | 65 | 0 | −6 | 6 |
| | hsa-miR-1250 | hsa-miR-1250 | 0 | 199 | 21 | −8 | 3 |
| | hsa-miR-1226 | hsa-miR-1226-3p | 0 | 153 | 55 | −7 | 1 |
| | hsa-miR-18a | hsa-miR-18a-3p | 0 | 143 | 31 | −7 | 2 |
| | hsa-miR-4677 | hsa-miR-4677-3p | 0 | 111 | 35 | −7 | 2 |
| | hsa-miR-513c | hsa-miR-513c-5p | 0 | 107 | 15 | −7 | 3 |
| | hsa-miR-138 | hsa-miR-138-2-3p | 0 | 99 | 29 | −7 | 2 |
| | hsa-miR-548ah | hsa-miR-548ah-3p | 0 | 99 | 29 | −7 | 2 |
| | hsa-miR-505 | hsa-miR-505-5p | 0 | 87 | 24 | −6 | 2 |
| | hsa-miR-193b | hsa-miR-193b-5p | 0 | 60 | 27 | −6 | 1 |
| | hsa-miR-18b | hsa-miR-18b-5p | 0 | 46 | 26 | −6 | 1 |
| Group 6 | hsa-miR-3117 | hsa-miR-3117-3p | 0 | 132 | 659 | −7 | −2 |
| 5 off in GON | hsa-miR-30b | hsa-miR-30b-3p | 0 | 124 | 747 | −7 | −3 |
| RT >> ON | hsa-miR-105 | hsa-miR-105-5p | 0 | 111 | 648 | −7 | −3 |
| | hsa-miR-19b | hsa-miR-19b-1-5p | 0 | 71 | 152 | −6 | −1 |
| | hsa-miR-376a | hsa-miR-376a-5p | 0 | 67 | 168 | −6 | −1 |

Group 1 and 2: 11 microRNA elevated in GON.
Group 3 and 4: 11 microRNA decreased in GON.
Group 5 and 6: 16 microRNA present in ON and absent or very low in GON.
microRNA names, miRbase [38], Ambros, et al., 2002 [39].

TABLE 6
18 microRNAs differentially regulated in glaucoma vs normal optic nerve and targeting HPG genes, with name,
mature arm, and strongest differential expression, evaluated through maximum and total expression levels.

| | | | Max GON | Max NON | log2 GON/ | Total ON | Total RT | log2 |
|---|---|---|---|---|---|---|---|---|
| | microRNA | Stronger arm | level | level | NON | levels | levels | NON/NRT |
| Group 1 | hsa-let-7c | hsa-let-7c | 263,645 | 188,920 | 0.48 | 811,062 | 174,457 | 2.22 |
| 13 up | hsa-miR-1248 | hsa-miR-1248 | 2,582 | 1,069 | 1.27 | 5,046 | 708 | 2.83 |
| GON>>ON | hsa-miR-574 | hsa-miR-574-5p | 956 | 660 | 0.53 | 2,845 | 210 | 3.75 |
| ON >> RT | hsa-miR-27a | hsa-miR-27a-5p | 161 | 66 | 1.27 | 271 | 24 | 3.44 |
| | hsa-miR-145 | hsa-miR-145-3p | 116 | 75 | 0.62 | 230 | 0 | 7.85 |
| | hsa-miR-5584 | hsa-miR-5584-5p | 97 | 71 | 0.44 | 229 | 0 | 7.84 |
| | hsa-let-7a-2 | hsa-let-7a-2-3p | 107 | 55 | 0.95 | 169 | 25 | 2.71 |
| | hsa-miR-549 | hsa-miR-549 | 161 | 0 | 7.34 | 129 | 0 | 7.02 |
| | hsa-miR-675 | hsa-miR-675-3p | 161 | 0 | 7.34 | 129 | 0 | 7.02 |
| | hsa-miR-148a | hsa-miR-148a-3p | 42,129 | 26,930 | 0.65 | 107,405 | 81,283 | 0.4 |
| | hsa-miR-455 | hsa-miR-455-5p | 1,203 | 616 | 0.96 | 3,180 | 2,419 | 0.39 |
| | hsa-miR-31 | hsa-miR-31-5p | 10,311 | 5,767 | 0.84 | 26,198 | 77,611 | −1.57 |
| | hsa-miR-216a | hsa-miR-216a | 524 | 330 | 0.67 | 1,355 | 5,990 | −2.14 |
| Group 2 | hsa-miR-181b | hsa-miR-181b-5p | 96,524 | 115,563 | −0.26 | 400,963 | 283,331 | 0.5 |
| 4 down | hsa-miR-545 | hsa-miR-545-5p | 0 | 83 | −6.39 | 110 | 25 | 2.1 |
| GON << ON | hsa-miR-3622a | hsa-miR-3622a-5p | 0 | 97 | −6.61 | 112 | 16 | 2.73 |
| ON >> RT | hsa-miR-548ah | hsa-miR-548ah-5p | 0 | 69 | −6.13 | 86 | 47 | 0.86 |

Group 1: 14 microRNA elevated in GON, lower or absent in RT.
Group 2: 4 microRNA decreased in GON, lower or absent in RT.

### DISCUSSION

Primary open-angle glaucoma (POAG) is clinically and genetically complex and enigmatic. Clinically, it is usually bilateral, though it may be asymmetric. People develop elevated intraocular pressure (IOP) due to disturbed aqueous humor dynamics. They have hampered outflow from the eye of the nutrient-containing aqueous humor. This is associated with nearly constant rate of aqueous production, no matter what the steady state IOP. Sustained, above-normal levels of IOP constitute the largest risk factor for developing characteristic damage to visual function, the clinical basis for glaucoma diagnosis. This damage affects the retinal ganglion cells, their axons, and the optic nerve in a diagnostic manner. Of clinical interest, not all eyes that have sustained elevated IOP develop damaged visual function (this is called ocular hypertension); and not all eyes that develop characteristic glaucomatous visual function damage have elevated IOP. Ten percent of untreated patients included in the Ocular Hypertension Treatment Study, enrolled with a sustained IOP elevation of 32 mmHg or less, developed glaucoma within 5 years, and 90 percent did not [40]. Thus, structures in the front of the eye and different structures in the posterior pole of the eye and its optic nerve are clinically separately impacted. This suggests the majority of high-pressure POAG (HPG) cases involve separate sets of causative gene alterations, one set for the anterior segment and the other set for the posterior segment of the eye. In addition to this, a history of POAG in a close family member doubles the risk for a person developing the disease.

In the study, genome-wide exome sequencing was used to investigate DNA variants in exons genome-wide from 295 Caucasian, high-pressure POAG patients whose genomes were previously evaluated in the NEIGHBOR GWAS. Our analysis strategy minimized false positive observations, focused on single nucleotide polymorphisms (SNPs), and compared frequencies of variants found in the POAG cases to frequencies in the ESP and 1000G databases. Further analysis of SNPs with POAG frequencies that differed significantly from 1000G or ESP identified genes of interest, grouped by number of SNPs with frequency differences and maximum frequency difference.

This study shows that in the part of the genome sequence containing exons and nearby bases, we found nearly 3 million SNP sites with bases different from the hg19 reference genome in three or more HPG patients. These sites were also found as SNP sites in the comparative general population databases, specifically, in 1000G Phase 1, in the European subset of 1000G Phase 3, in ESP, and in the European subset of ESP. The HPG variant sites were calculated directly from the sequence data and then compared. The high level of consistency with the public databases indicates the alignment and variant calling methods used to process patient sequence data were accurate.

For all sites that differed from hg19 in three or more patients, we revisited the exome sequence data, and for every patient inspected whether the data supported each possible allele, including the reference hg19 base, the most frequent alternative (non-hg19) allele, and any additional allele observed by us or others at that site. We calculated that fewer than 5% of the hg19 reference bases in 1000G SNP sites were the minor, less frequent allele. Since genotype calls for those sites using exome sequence data may be biased toward the minor (same as hg19) allele, we set those sites aside for future consideration. We further calculated what minimum number of patient observations at a given site would be needed to obtain allele frequencies between 0.00 and 1.00 with 0.02 increments; that number was 25 patients. So we further set aside any SNP site that was measured in fewer than 25 patients.

When we compared the HPG patient minor allele frequencies for each remaining site with the 1000G database frequencies for that site, we noted, when the filters were applied, the HPG patients had a number of sites where the general population minor allele was over-represented in HPG. These sites provide pointers to locations within the genome where polymorphisms occur at disproportionate rates in HPG patients. Next, having identified how many HPG had the minor allele in comparison with the normative database, we were able to identify the SNP sites that are vastly overrepresented in HPG.

Using a minimum of 0.10 frequency difference between HPG patients and general population databases, requiring a minimum of 25 HPG patients with observations at a given site, and considering only sites within or near genes and expressed in ocular tissues, 933 SNP sites were retained for statistical analysis. Requiring the odds ratio 95% confidence interval lower bound be above 1.0, HPG frequency exceed least squares fit to data, and p-value remain significant after Bonferroni correction for multiple testing, 160 sites in 140 genes remained.

We compared the 140 genes to lists of genes previously implicated in glaucoma, neurological diseases, or other eye diseases and to lists of genes involved in inflammatory response, cell adhesion, or expression in trabecular meshwork and obtained annotations for 85 of 140 genes.

This is the first investigation of the actual exome of HPG patients. This contrasts with array-based association studies that looked for markers primarily outside the exome. The sites investigated here are all within gene regulatory or coding regions. Not all genes were sequenced in sufficient depth here for full consideration. For example, the CDKN2B/CDKN2B-AS1 association found through array-based GWAS was not replicated here because the associated sites were not sequenced. There may be other genes with a similar status.

Clinical filters were used here for discovery. Prior studies that used exome sequence data for genome-wide association used p-values as the discriminating criterion, some in a burden test and some through classic association tests. It would be of interest to return to the data in these other studies with the clinical criteria used here.

In the current study, the majority of the sites identified as associated with disease are located in the regulatory regions of the genome adjacent to coding regions. Schork et al, PLoS Genet. 2013 April; 9(4):e1003449 [41] noted that for associations in traditional GWAS, imputation indicates SNPs in untranslated regions and proximal promoters are over-represented, consistent with our findings here through direct exome sequencing. While it is entirely possible that SNPs involved in glaucoma with high pressure are located outside these regulatory regions, as in CDKN2B/CDKN2B-AS1, this study is the first deep sequencing analysis of regulatory exons and proximal promoter and intron bases.

The first gene linked to glaucoma was Myocilin (MYOC). We measured all bases in MYOC in patients, and no single site passed our filters. MYOC was linked to glaucoma through family studies and was primarily related to juvenile onset open-angle glaucoma. MYOC mutations are present in only about 3-4% of adult POAG, as reported in Alward et al., Arch. Ophthalmology. (2002) 120(9):1189-97 [42], and reviewed in Fingert el al., Surv. Ophthalmology. (2002) 47(6):547-61 [43].

Similarly, we would not expect to find SNPs associated with the optineurin gene (OPTN) in our investigation since it has been found to be associated with normal tension glaucoma, not with HPG, as reported in Rezaie et al., Science. (2002) 295(5557):1077-9 [44].

For some SNP sites, a high percentage, greater than 80%, of HPG patients had the minor allele compared with a much smaller fraction of the normative databases. This finding may point to a few select genes, whose polymorphisms are heavily represented in the HPG patients.

In this study, we confined our attention to sequencing and analyzing the exome in self-reported Caucasian, European-background HPG patients. Our sequencing included the exons, their UTRs, and nearby bases in the introns. After sequencing, comparison with the hg19 reference database disclosed a huge number, many millions, of SNP sites in our HPG patients, any one, or more, of which might explain HPG. To find the HPG sites and the associated genes has been the goal. To identify sites related to disease, we developed a clinically intuitive, serial procedure to identify, in comparison with general population databases, e.g., the ESP and 1000 Genome databases, a workable number of candidate SNP sites in HPG patients. This method provides a path to a list of associated, potentially causative disease genes that can be used to predict onset, progression, severity, or recurrence of disease after treatment. Additional work will require assessment of the role of candidate genes in the anterior and posterior segments of the eye. Further, the sites and their genes can be considered in doublets or higher numbers of interacting mutations that affect the eye and cause HPG.

This investigation identified, and categorized, SNP-containing genes present in unusually high frequency in HPG patients compared with the general population.

In summary, we found 140 genes associated with and/or causative of HPG and appropriate for predicting onset, progression, or severity of disease or recurrence after treatment. The vast majority of the 140 genes were not previously associated with HPG. These genes were found selectively in HPG compared to general and European population datasets.

Five of the 140 genes identified in the present study were previously associated with glaucoma. This study shows that the 135 newly associated genes and the five previously associated genes all have variants with highly elevated frequencies in HPG.

### REFERENCES

1. Quigley H A, Broman A T. The number of people with glaucoma worldwide in 2010 and 2020. Br J Ophthalmol. 2006 March; 90(3):262-7. PubMed PMID: 16488940; PubMed Central PMCID: PMC1856963.
2. Tielsch J M, Sommer A, Katz J, Royall R M, Quigley H A, Javitt J. Racial variations in the prevalence of primary open-angle glaucoma. The Baltimore Eye Survey. JAMA. 1991 Jul. 17; 266(3):369-74. PubMed PMID: 2056646.
3. The AGIS Investigators. The Advanced Glaucoma Intervention Study (AGIS): 7. The relationship between control of intraocular pressure and visual field deterioration. Am J Ophthalmol. 2000 October; 130(4):429-40. PubMed PMID: 11024415.
4. Anderson D R. Glaucoma: the damage caused by pressure. XLVI Edward Jackson memorial lecture. Am J Ophthalmol. 1989 Nov. 15; 108(5):485-95. Review. PubMed PMID: 2683792.
5. Mitchell P, Rochtchina E, Lee A J, Wang J J. Bias in self-reported family history and relationship to glaucoma: the Blue Mountains Eye Study. Ophthalmic Epidemiol. 2002 December; 9(5):333-45. PubMed PMID: 12528918.
6. Tielsch J M, Katz J, Sommer A, Quigley H A, Javitt J C. Family history and risk of primary open angle glaucoma. The Baltimore Eye Survey. Arch Ophthalmol. 1994 January; 112(1):69-73. PubMed PMID: 8285897.
7. Sommer A, Tielsch J M, Katz J, Quigley H A, Gottsch J D, Javitt J, Singh K. Relationship between intraocular pressure and primary open angle glaucoma among white and black Americans. The Baltimore Eye Survey. Arch Ophthalmol. 1991 August; 109(8): 1090-5. PubMed PMID: 1867550.
8. Wiggs J L, Yaspan B L, Hauser M A, Kang J H, Allingham R R, Olson L M, Abdrabou W, Fan B J, Wang D Y, Brodeur W, Budenz D L, Caprioli J, Crenshaw A, Crooks K, Delbono E, Doheny K F, Friedman D S, Gaasterland D, Gaasterland T, Laurie C, Lee R K, Lichter P R, Loomis S, Liu Y, Medeiros F A, McCarty C, Mirel D, Moroi S E, Musch D C, Realini A, Rozsa F W, Schuman J S, Scott K, Singh K, Stein J D, Trager E H, Vanveldhuisen P, Vollrath D, Wollstein G, Yoneyama S, Zhang K, Weinreb R N, Ernst J, Kellis M, Masuda T, Zack D, Richards J E, Pericak-Vance M, Pasquale L R, Haines J L. Common variants at 9p21 and 8q22 are associated with increased susceptibility to optic nerve degeneration in glaucoma. PLoS Genet. 2012; 8(4):e1002654. doi: 10.1371/journal.pgen.1002654. Epub 2012 Apr. 26. PubMed PMID: 22570617; PubMed Central PMCID: PMC3343074.
9. Fan B J, Wang D Y, Pasquale L R, Haines J L, Wiggs J L. Genetic variants associated with optic nerve vertical cup-to-disc ratio are risk factors for primary open angle glaucoma in a US Caucasian population. Invest Ophthalmol Vis Sci. 2011 Mar. 28; 52(3):1788-92. doi: 10.1167/iovs.10-6339. PubMed PMID: 21398277; PubMed Central PMCID: PMC3101676.
10. Thorleifsson G, Magnusson K P, Sulem P, Walters G B, Gudbjartsson D F, Stefansson H, Jonsson T, Jonasdottir A, Jonasdottir A, Stefansdottir G, Masson G, Hardarson G A, Petursson H, Arnarsson A, Motallebipour M, Wallerman O, Wadelius C, Gulcher J R, Thorsteinsdottir U, Kong A, Jonasson F, Stefansson K. Common sequence variants in the LOXL1 gene confer susceptibility to exfoliation glaucoma. Science. 2007 Sep. 7; 317(5843):1397-400. Epub 2007 Aug. 9. PubMed PMID: 17690259.
11. Thorleifsson G, Walters G B, Hewitt A W, Masson G, Helgason A, DeWan A, Sigurdsson A, Jonasdottir A, Gudjonsson S A, Magnusson K P, Stefansson H, Lam D S, Tam P O, Gudmundsdottir G J, Southgate L, Burdon K P, Gottfredsdottir M S, Aldred M A, Mitchell P, St Clair D, Collier D A, Tang N, Sveinsson O, Macgregor S, Martin N G, Cree A J, Gibson J, Macleod A, Jacob A, Ennis S, Young T L, Chan J C, Karwatowski W S, Hammond C J, Thordarson K, Zhang M, Wadelius C, Lotery A J, Trembath R C, Pang C P, Hoh J, Craig J E, Kong A, Mackey D A, Jonasson F, Thorsteinsdottir U, Stefansson K. Common variants near CAV1 and CAV2 are associated with primary open-angle glaucoma. Nat Genet. 2010 October; 42(10):906-9. doi: 10.1038/ng.661. Epub 2010 Sep. 12. PubMed PMID: 20835238; PubMed Central PMCID: PMC3222888.
12. Burdon K P, Macgregor S, Hewitt A W, Sharma S, Chidlow G, Mills R A, Danoy P, Casson R, Viswanathan A C, Liu J Z, Landers J, Henders A K, Wood J, Souzeau E, Crawford A, Leo P, Wang J J, Rochtchina E, Nyholt D R, Martin N G, Montgomery G W, Mitchell P, Brown M A, Mackey D A, Craig J E. Genome-wide association study identifies susceptibility loci for open angle glaucoma at TMCO1 and CDKN2B-AS1. Nat Genet. 2011 June; 43(6):574-8. doi: 10.1038/ng.824. Epub 2011 May 1. PubMed PMID: 21532571.
13. Nowak A, Majsterek I, Przybylowska-Sygut K, Pytel D, Szymanek K, Szaflik J, Szaflik J P. Analysis of the Expression and Polymorphism of APOE, HSP, BDNF, and GRIN2B Genes Associated with the Neurodegeneration Process in the Pathogenesis of Primary Open Angle Glaucoma. Biomed Res Int. 2015; 2015:258281. doi: 10.1155/2015/258281. Epub 2015 Mar. 29. PubMed PMID: 25893192; PubMed Central PMCID: PMC4393917.
14. Nowak A, Szaflik J P, Gacek M, Przybylowska-Sygut K, Kamińska A, Szaflik J, Majsterek I. BDNF and HSP gene polymorphisms and their influence on the progression of primary open-angle glaucoma in a Polish population. Arch Med Sci. 2014 Dec. 22; 10(6):1206-13. doi: 10.5114/aoms.2014.45089. Epub 2014 Sep. 5. PubMed PMID: 25624860; PubMed Central PMCID: PMC4296062.
15. Psychiatric GWAS Consortium Coordinating Committee, Cichon S, Craddock N, Daly M, Faraone S V, Gejman P V, Kelsoe J, Lehner T, Levinson D F, Moran A, Sklar P, Sullivan P F. Genomewide association studies: history, rationale, and prospects for psychiatric disorders. Am J Psychiatry. 2009 May; 166(5):540-56. doi: 10.1176/appi.ajp.2008. Ser. No. 08/091,354. Epub 2009 Apr. 1. Review. PubMed PMID: 19339359; PubMed Central PMCID: PMC3894622.
16. Nho K, Shen L, Kim S, Swaminathan S, Risacher S L, Saykin A J; Alzheimer's Disease Neuroimaging Initiative (ADNI). The effect of reference panels and software tools on genotype imputation. AMIA Annu Symp Proc. 2011; 2011:1013-8. Epub 2011 Oct. 22. PubMed PMID: 22195161; PubMed Central PMCID: PMC3243280.
17. Lee S, Abecasis G R, Boehnke M, Lin X. Rare-variant association analysis: study designs and statistical tests. Am J Hum Genet. 2014 Jul. 3; 95(1):5-23. doi: 10.1016/j.ajhg.2014.06.009. Review PubMed PMID: 24995866; PubMed Central PMCID: PMC4085641.
18. Zuk O, Schaffner S F, Samocha K, Do R, Hechter E, Kathiresan S, Daly M J, Neale B M, Sunyaev S R, Lander E S. Searching for missing heritability: designing rare variant association studies. Proc Natl Acad Sci USA. 2014 Jan. 28; 111(4):E455-64. doi: 10.1073/pnas.1322563111. Epub 2014 Jan. 17. PubMed PMID: 24443550; PubMed Central PMCID: PMC3910587.
19. Nalls M A, Pankratz N, Lill C M, Do C B, Hernandez D G, Saad M, DeStefano A L, Kara E, Bras J, Sharma M, Schulte C, Keller M F, Arepalli S, Letson C, Edsall C, Stefansson H, Liu X, Pliner H, Lee J H, Cheng R; International Parkinson's Disease Genomics Consortium (IPDGC); Parkinson's Study Group (PSG) Parkinson's Research: The Organized GENetics Initiative (PROGENI); 23andMe; GenePD; NeuroGenetics Research Consortium (NGRC); Hussman Institute of Human Genomics (HIHG); Ashkenazi Jewish Dataset Investigator; Cohorts for Health and Aging Research in Genetic Epidemiology (CHARGE); North American Brain Expression Consortium (NABEC); United Kingdom Brain Expression Consortium (UKBEC); Greek Parkinson's Disease Consortium; Alzheimer Genetic Analysis Group, Ikram M A, Ioannidis J P, Hadjigeorgiou G M, Bis J C, Martinez M, Perlmutter J S, Goate A, Marder K, Fiske B, Sutherland M, Xiromerisiou G, Myers R H, Clark L N, Stefansson K, Hardy J A, Heutink P, Chen H, Wood N W, Houlden H, Payami H, Brice A, Scott W K, Gasser T, Bertram L, Eriksson N, Foroud T, Singleton A B. Large-scale meta-analysis of genome-wide association data identifies six new risk loci for Parkinson's disease. Nat Genet. 2014 September; 46(9):989-93. doi: 10.1038/ng.3043. Epub 2014 Jul. 27. PubMed PMID: 25064009; PubMed Central PMCID: PMC4146673.
20. Ng P C, Kirkness E F. Whole genome sequencing. Methods Mol Biol. 2010; 628:215-26. doi: 10.1007/978-1-60327-367-1_{—}12. Review. PubMed PMID: 20238084.
21. Fu W, O'Connor T D, Jun G, Kang H M, Abecasis G, Leal S M, Gabriel S, Rieder M J, Altshuler D, Shendure J, Nickerson D A, Bamshad M J; NHLBI Exome Sequencing Project, Akey J M. Analysis of 6,515 exomes reveals the recent origin of most human protein-coding variants. Nature. 2013 Jan. 10; 493(7431):216-20. doi: 10.1038/nature11690. Epub 2012 Nov. 28. Erratum in: Nature. 2013 Mar. 14; 495(7440):270. Rieder, Mark J [added]. PubMed PMID: 23201682; PubMed Central PMCID: PMC3676746.
22. Volk A, Conboy E, Wical B, Patterson M, Kirmani S. Whole-Exome Sequencing in the Clinic: Lessons from Six Consecutive Cases from the Clinician's Perspective. Mol Syndromol. 2015 February; 6(1):23-31. doi: 10.1159/000371598. Epub 2015 Feb. 3. Review. PubMed PMID: 25852444; PubMed Central PMCID: PMC4369115.
23. Ederer F, Gaasterland D E, Sullivan E K; AGIS Investigators. The Advanced Glaucoma Intervention Study (AGIS): 1. Study design and methods and baseline characteristics of study patients. Control Clin Trials. 1994 August; 15(4):299-325.PubMed PMID: 7956270.
24. van Koolwijk L M, Bunce C, Viswanathan A C. Gene finding in primary open-angle glaucoma. J Glaucoma. 2013 August; 22(6):473-86. doi: 10.1097/IJG.0b013e318255bc37. Review. PubMed PMID: 22549476.
25. Burdon K P. Genome-wide association studies in the hunt for genes causing primary open-angle glaucoma: a review. Clin Experiment Ophthalmol. 2012 May-June; 40(4):358-63. doi: 10.1111/j.1442-9071.2011.02744.x. Epub 2012 Feb. 20. Review. PubMed PMID: 22171998.
26. Allingham R R, Liu Y, Rhee D J. The genetics of primary open-angle glaucoma: a review. Exp Eye Res. 2009 April; 88(4):837-44. doi: 10.1016/j.exer.2008.11.003. Epub 2008 Nov. 14. Review. PubMed PMID: 19061886.
27. Wiggs J L, Hauser M A, Abdrabou W, Allingham R R, Budenz D L, Delbono E, Friedman D S, Kang J H, Gaasterland D, Gaasterland T, Lee R K, Lichter P R, Loomis S, Liu Y, McCarty C, Medeiros F A, Moroi S E, Olson L M, Realini A, Richards J E, Rozsa F W, Schuman J S, Singh K, Stein J D, Vollrath D, Weinreb R N, Wollstein G, Yaspan B L, Yoneyama S, Zack D, Zhang K, Pericak-Vance M, Pasquale L R, Haines J L. The NEIGHBOR consortium primary open-angle glaucoma genome-wide association study: rationale, study design, and clinical variables. J Glaucoma. 2013 September; 22(7):517-25. doi: 10.1097/IJG.0b013e31824d4fd8. PubMed PMID: 22828004; PubMed Central PMCID: PMC3485429.
28. Karolchik D, Barber G P, Casper J, Clawson H, Cline M S, Diekhans M, Dreszer T R, Fujita P A, Guruvadoo L, Haeussler M, Harte R A, Heitner S, Hinrichs A S, Learned K, Lee B T, Li C H, Raney B J, Rhead B, Rosenbloom K R, Sloan C A, Speir M L, Zweig A S, Haussler D, Kuhn R M, Kent W J. The UCSC Genome Browser database: 2014 update. Nucleic Acids Res. 2013 Nov. 21. [Epub ahead of print] PubMed PMID: 24270787.
29. Langmead B, Salzberg S L. Fast gapped-read alignment with Bowtie 2. Nat Methods. 2012 Mar. 4; 9(4):357-9. doi: 10.1038/nmeth.1923. PubMed PMID: 22388286; PubMed Central PMCID: PMC3322381.
30. Li H.*, Handsaker B.*, Wysoker A., Fennell T., Ruan J., Homer N., Marth G., Abecasis G., Durbin R. and 1000 Genome Project Data Processing Subgroup (2009) The Sequence alignment/map (SAM) format and SAMtools. Bioinformatics, 25, 2078-9. PubMed PMID: 19505943.
31. Seattleseq Annotation Server, Seattle, Wash. (URL: http://snp.gs.washington.edu/SeattleSeqAnnotation137) [September-December 2013]
32. Ng S B, Turner E H, Robertson P D, Flygare S D, Bigham A W, Lee C, Shaffer T, Wong M, Bhattacharjee A, Eichler E E, Bamshad M, Nickerson D A, Shendure J. Targeted capture and massively parallel sequencing of 12 human exomes. Nature. 2009 Sep.10; 461(7261):272-6. doi: 10.1038/nature08250. Epub 2009 Aug. 16. PubMed PMID: 19684571; PubMed Central PMCID: PMC2844771.
33. Adzhubei I A, Schmidt S, Peshkin L, Ramensky V E, Gerasimova A, Bork P, Kondrashov A S, Sunyaev S R. A method and server for predicting damaging missense mutations. Nat Methods. 2010 April; 7(4):248-9. doi: 10.1038/nmeth0410-248. PubMed PMID: 20354512; PubMed Central PMCID: PMC2855889.
34. Sim N L, Kumar P, Hu J, Henikoff S, Schneider G, Ng P C. SIFT web server: predicting effects of amino acid substitutions on proteins. Nucleic Acids Res. 2012 July; 40(Web Server issue):W452-7. doi: 10.1093/nar/gks539. Epub 2012 Jun. 11. PubMed PMID: 22689647; PubMed Central PMCID: PMC3394338.
35. 1000 Genomes Project Consortium, Abecasis G R, Auton A, Brooks L D, DePristo M A, Durbin R M, Handsaker R E, Kang H M, Marth G T, McVean G A. An integrated map of genetic variation from 1,092 human genomes. Nature. 2012 Nov. 1; 491(7422):56-65. doi: 10.1038/nature11632. PubMed PMID: 23128226; PubMed Central PMCID: PMC3498066.
36. Exome Variant Server, NHLBI GO Exome Sequencing Project (ESP), Seattle, Wash. (URL: http://evs.gs.washington.edu/EVS/) [September-December, 2013].
37. Human Gene Nomenclature Committee, genenames.org (Gene Identifier Table from ftp://ftp.ebi.ac.uk/pub/databases/genenames/new/tsv/hgnc_complete_set.txt)
38. mirBase, mirbase.org (microRNA identifiers with matures sequences from ftp://mirbase.org/pub/mirbase/CURRENT/mature.fa.gz)
39. Ambros V, Bartel B, Bartel D P, Burge C B, Carrington J C, Chen X, Dreyfuss G, Eddy S R, Griffiths-Jones S, Marshall M, Matzke M, Ruvkun G, Tuschl T. A uniform system for microRNA annotation. RNA. 2003 March; 9(3):277-9. PubMed PMID: 12592000; PubMed Central PMCID: PMC1370393.
40. Kass M A, Heuer D K, Higginbotham E J, Johnson C A, Keltner J L, Miller J P, Parrish R K 2nd, Wilson M R, Gordon M O. The Ocular Hypertension Treatment Study: a randomized trial determines that topical ocular hypotensive medication delays or prevents the onset of primary open-angle glaucoma. Arch Ophthalmol. 2002 June; 120(6):701-13; discussion 829-30. PubMed PMID: 12049574.
41. Schork A J, Thompson W K, Pham P, Torkamani A, Roddey J C, Sullivan P F, Kelsoe J R, O'Donovan M C, Furberg H; Tobacco and Genetics Consortium; Bipolar Disorder Psychiatric Genomics Consortium; Schizophrenia Psychiatric Genomics Consortium, Schork N.J., Andreassen O A, Dale A M. All SNPs are not created equal: genome-wide association studies reveal a consistent pattern of enrichment among functionally annotated SNPs. PLoS Genet. 2013 April; 9(4):e1003449. doi: 10.1371/journal.pgen.1003449. Epub 2013 Apr. 25. PubMed PMID: 23637621; PubMed Central PMCID: PMC3636284
42. Alward W L, Kwon Y H, Khanna C L, Johnson A T, Hayreh S S, Zimmerman M B, Narkiewicz J, Andorf J L, Moore P A, Fingert J H, Sheffield V C, Stone E M. Variations in the myocilin gene in patients with open-angle glaucoma. Arch Ophthalmol. 2002 September; 120(9):1189-97. PubMed PMID: 12215093.
43. Fingert J H, Stone E M, Sheffield V C, Alward W L. Myocilin glaucoma. Sury Ophthalmol. 2002 November-December; 47(6):547-61. Review. PubMed PMID: 12504739.
44. Rezaie T, Child A, Hitchings R, Brice G, Miller L, Coca-Prados M, Heon E, Krupin T, Ritch R, Kreutzer D, Crick R P, Sarfarazi M. Adult-onset primary open-angle glaucoma caused by mutations in optineurin. Science. 2002 Feb. 8; 295(5557):1077-9. PubMed PMID: 11834836.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of identifying genes whose alleles are associative with or causative of the onset and/or progression and/or severity and/or recurrence of a disease, comprising:
a) sequencing or reviewing multiple exomes from patients who have been diagnosed with the disease and one or more exomes from one or more individuals known not to have the disease, wherein the one or more exomes from one or more individuals known not to have the disease comprise one or more reference exomes;
b) selecting exomes sequenced and read with a fidelity of 4 or fewer mismatches per 100 bases;
c) selecting for genes having one or more site variants in the exomes from patients who have been diagnosed with the disease with one or more properties, for example, 1, 2, 3, 4, 5, 6, 7, 8, 9, 10, 11, 12, 13, 14, 15, 16, 17, or 18 properties, selected from:
i) site variant is found in one or more patients;
ii) site variant is observed in a general population dataset;
iii) site variant is found in three or more patients;
iv) one or more reference exomes have the major allele;
v) site variant is the minor allele in reference exomes;
vi) site variant has only one alternate allele;
vii) site is within genome region with balanced G+C and A+T content;
viii) site is located outside low complexity genome regions;
ix) site is located in genome region with no paralog within 95% identity; and
x) site variant is located on chromosomes 1-22 or site variant is located on chromosome X or Y only if disease incidence is gender-biased;
xi) site was measured in 25 or more patients;
xii) site variant frequency in patients differs from general populations by more than expected measurement error, e.g., 0.05 (on a frequency scale from 0.00-1.00);
xiii) site variant frequency in patients exceeds general populations, e.g., by more than 0.10;
xiv) site variant is within a gene or regulatory regions influencing its expression as RNA or protein;
xv) site variant is within or near a gene expressed in tissues relevant to disease;
xvi) odds ratio 95% confidence interval lower bound calculated for the site from patient and reference general population frequencies is above 1.00;
xvii) frequency of site variant in patients is above a line fitted to filtered sites represented as datapoints where X is reference general population frequency and Y is patient frequency, e.g., fit with least squares linear regression; and
xviii) a p-value calculated with a 2×2 statistical test, e.g., Fisher's Exact Test, from numbers of alternate and reference alleles observed for the site in patients and in general population remains significant after correction for multiple testing.

2. The method of claim 1, wherein the disease is a neurodegenerative disease, cancer, a cardiovascular disease, an immune disease, an autoimmune disease, an endocrinologic disease, or an inflammatory disease.

3. The method of claim 1, wherein the disease is a neurodegenerative disease.

4. The method of claim 3, wherein the disease is an ocular disease.

5. The method of claim 4, wherein the disease is primary open angle glaucoma (POAG).

6. The method of claim 1, wherein the patients are symptomatic for the disease.

7. The method of claim 1, wherein the method is computer implemented.

8. The method of claim 1, wherein the site variants are selected from single nucleotide polymorphisms (SNPs), insertions, deletions and rearrangements.

9. The method of claim 1, further comprising determining the expression levels of the genes from patient exomes and reference exomes.

10. The method of claim 1, further comprising determining the expression levels of the microRNA from patient exomes and reference exomes.

11. The method of claim 1, wherein sequencing comprises employing a next-generation sequencing (NGS) technique or method.

12. The method of claim 1, comprising selecting exomes sequenced and read with a fidelity of 3 or fewer mismatches per 100 bases.

13. The method of claim 1, wherein the general population comparison dataset is selected from one or more of 1000 Genomes, the Exome Sequencing Project datasets, UK10K, UCSC Genome Bioinformatics Site, and/or other available public and proprietary datasets.

14. The method of claim 1, further comprising weighting said selected genes according to predictive power rankings of the collection of signature biomarkers.

15. A method for predicting onset and/or progression and/or severity and/or recurrence of primary open angle glaucoma (POAG) in a subject, the method comprising:
(a) receiving allelic information and/or expression levels of a collection of signature biomarkers from a biological sample taken from said subject suspected of developing or suffering POAG, wherein said collection of signature biomarkers comprises one or more genes and/or microRNA selected from the group consisting of: AATF, ABI1, ABI3BP, ACTN2, ADAMTS15, ADCY2, AHNAK2, ANGEL2, ANKRD36, ANKRD36B, ANO5, AP1M1, ARHGAP30, ASTN1, ATP6V1E2, BAI3, CACNA1E, CACNA1I, CALM1, CCDC66, CD163, CDH13, CDH4, CDK17, CELF5, CHD8, CLCA4, CLEC7A, CLSTN2, CNNM2, CNOT6, COL23A1, COL4A2, CRTAC1, CTU2, CYBA, DCBLD2, DHCR7, DNAJB11, DPF3, DRD2, EBF2, ENO3, EPT1, ERI2, FDX1L, FJ22184, FOXD4, FOXRED2, FRYL, GAS7, GNG7, GOLGA3, GRIA1, GRID1, GRM4, HERC2, HLA-A, HLA-DRB1, IFI6, IMMT, INPP5D, ITGB4, KIAA0930, LACTB2, LCP2, LEMD3, LILRB2, LILRB3, LIN7A, LOC642846, LOC643387, LOC728537, LPHN3, LRP3, LRP4, LRRC37A, MAML3, MATR3, MCCC1, MCF2L, MEGF11, MGC21881, MINK1, MRPL23, MUC4, MYH9, MYO1E, N6AMT1, NBPF16, NOMO2, NUCKS1, PALM2, PCK1, PCM1, PDE4DIP, PML, POTEC, PPFIA2, PRKAG2, PRKCH, PRKD1, PRUNE2, R3HDM1, RABGAP1, RAD51B, RBFOX1, RIN3, SARDH, SCAF8, SEC14L1, SEL1L3, SEMA5A, SEMA5B, SIRT1, SLC30A8, SNTB1, SPN, SPRY1, SRRM2, TMPRSS13, TNRC18, TOR1A, TRIM58, TSPAN11, TXNRD1, UNC5B, USP20, USP6, VAC14, VARS2, VCAN, WASH1, XRCC5, ZDHHC7, ZMYND11, ZNF155, ZNF573, ZNF594, ZNF83, hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, hsa-miR-99b-5p, hsa-miR-1246, hsa-miR-1248, hsa-miR-130a, hsa-miR-130a-3p, hsa-miR-145, hsa-miR-145-3p, hsa-miR-148a, hsa-miR-148a-3p, hsa-miR-214, hsa-miR-214-3p, hsa-miR-216a, hsa-miR-224, hsa-miR-224-5p, hsa-miR-27a-5p, hsa-miR-31, hsa-miR-31-5p, hsa-miR-4448, hsa-miR-449a, hsa-miR-452, hsa-miR-452-5p, hsa-miR-455, hsa-miR-455-5p, hsa-miR-483, hsa-miR-483-3p, hsa-miR-483-5p, hsa-miR-549, hsa-miR-5584, hsa-miR-5584-5p, hsa-miR-574, hsa-miR-574-5p, hsa-miR-675, hsa-miR-675-3p, hsa-miR-767, hsa-miR-767-5p, hsa-miR-9, hsa-miR-9-3p, msa-miR-27a, hsa-let-7a, hsa-let-7a-2, hsa-let-7a-2-3p, and hsa-let-7c;
(b) applying the allelic information and/or expression levels to a predictive model relating allelic information and/or expression levels of said collection of signature biomarkers with onset of POAG; and (c) evaluating an output of said predictive model to predict onset of POAG in said individual; and/or
(c) applying the allelic information and/or expression levels to a predictive model relating allelic information and/or expression levels of said collection of signature biomarkers with progression of POAG; and (e) evaluating an output of said predictive model to predict progression of POAG in said individual; and/or
(d) applying the allelic information and/or expression levels to a predictive model relating allelic information and/or expression levels of said collection of signature biomarkers with severity of POAG; and (g) evaluating an output of said predictive model to predict severity of POAG in said individual; and/or
(e) applying the allelic information and/or expression levels to a predictive model relating allelic information and/or expression levels of said collection of signature biomarkers with recurrence of POAG; and (i) evaluating an output of said predictive model to predict recurrence of POAG in said individual.

16. The method of claim 15, wherein said collection of signature biomarkers comprises one or more genes selected from the group consisting of: AATF, ABI1, ABI3BP, ACTN2, ADAMTS15, ADCY2, AHNAK2, ANGEL2, ANKRD36, ANKRD36B, ANO5, AP1M1, ARHGAP30, ASTN1, ATP6V1E2, BAI3, CACNA1E, CACNA1I, CALM1, CCDC66, CD163, CDH13, CDH4, CDK17, CELF5, CHD8, CLCA4, CLEC7A, CLSTN2, CNNM2, CNOT6, COL23A1, COL4A2, CRTAC1, CTU2, CYBA, DCBLD2, DHCR7, DNAJB11, DPF3, DRD2, EBF2, ENO3, EPT1, ERI2, FDX1L, FLJ22184, FOXD4, FOXRED2, FRYL, GAS7, GNG7, GOLGA3, GRIA1, GRID1, GRM4, HERC2, HLA-A, HLA-DRB1, IFI6, IMMT, INPP5D, ITGB4, KIAA0930, LACTB2, LCP2, LEMD3, LILRB2, LILRB3, LIN7A, LOC642846, LOC643387, LOC728537, LPHN3, LRP3, LRP4, LRRC37A, MAML3, MATR3, MCCC1, MCF2L, MEGF11, MGC21881, MINK1, MRPL23, MUC4, MYH9, MYO1E, N6AMT1, NBPF16, NOMO2, NUCKS1, PALM2, PCK1, PCM1, PDE4DIP, PML, POTEC, PPFIA2, PRKAG2, PRKCH, PRKD1, PRUNE2, R3HDM1, RABGAP1, RAD51B, RBFOX1, RIN3, SARDH, SCAF8, SEC14L1, SEL1L3, SEMA5A, SEMA5B, SIRT1, SLC30A8, SNTB1, SPN, SPRY1, SRRM2, TMPRSS13, TNRC18, TOR1A, TRIM58, TSPAN11, TXNRD1, UNC5B, USP20, USP6, VAC14, VARS2, VCAN, WASH1, XRCC5, ZDHHC7, ZMYND11, ZNF155, ZNF573, ZNF594, and ZNF83 wherein the position and allele of the genetic variation associated with and/or causative of POAG is as provided in Table 4.

17. The method of claim 15, wherein said collection of signature biomarkers comprises one or more genes is selected from the group consisting of: COL4A2, COL23A1, GAS7, VCAN, and HLA-DRB1, wherein the position and allele of the genetic variation associated with and/or causative of POAG is as provided in Table 4.

18. The method of claim 15, wherein overexpression of one or more microRNAs selected from hsa-miR-1246, hsa-miR-1248, hsa-miR-130a, hsa-miR-130a-3p, hsa-miR-145, hsa-miR-145-3p, hsa-miR-148a, hsa-miR-148a-3p, hsa-miR-214, hsa-miR-214-3p, hsa-miR-216a, hsa-miR-224, hsa-miR-224-5p, hsa-miR-27a-5p, hsa-miR-31, hsa-miR-31-5p, hsa-miR-4448, hsa-miR-449a, hsa-miR-452, hsa-miR-452-5p, hsa-miR-455, hsa-miR-455-5p, hsa-miR-483, hsa-miR-483-3p, hsa-miR-483-5p, hsa-miR-549, hsa-miR-5584, hsa-miR-5584-5p, hsa-miR-574, hsa-miR-574-5p, hsa-miR-675, hsa-miR-675-3p, hsa-miR-767, hsa-miR-767-5p, hsa-miR-9, hsa-miR-9-3p, msa-miR-27a, hsa-let-7a, hsa-let-7a-2, hsa-let-7a-2-3p, and hsa-let-7c in the biological sample from the subject in comparison to a control sample from an individual known not to have POAG predicts a negative outcome or onset and/or progression and/or severity and/or recurrence of POAG.

19. The method of claim 18, further comprising administering to the subject an inhibitory nucleic acid that reduces or inhibits the expression of one or more microRNAs selected from hsa-miR-1246, hsa-miR-1248, hsa-miR-130a, hsa-miR-130a-3p, hsa-miR-145, hsa-miR-145-3p, hsa-miR-148a, hsa-miR-148a-3p, hsa-miR-214, hsa-miR-214-3p, hsa-miR-216a, hsa-miR-224, hsa-miR-224-5p, hsa-miR-27a-5p, hsa-miR-31, hsa-miR-31-5p, hsa-miR-4448, hsa-miR-449a, hsa-miR-452, hsa-miR-452-5p, hsa-miR-455, hsa-miR-455-5p, hsa-miR-483, hsa-miR-483-3p, hsa-miR-483-5p, hsa-miR-549, hsa-miR-5584, hsa-miR-5584-5p, hsa-miR-574, hsa-miR-574-5p, hsa-miR-675, hsa-miR-675-3p, hsa-miR-767, hsa-miR-767-5p, hsa-miR-9, hsa-miR-9-3p, msa-miR-27a, hsa-let-7a, hsa-let-7a-2, hsa-let-7a-2-3p, and hsa-let-7c.

20. The method of claim 18, further comprising administering to the subject one or more microRNAs or one or more mimics of microRNAs selected from hsa-miR-130a, hsa-miR-1246, hsa-miR-214, hsa-miR-452, hsa-miR-224, hsa-miR-4448, hsa-miR-483, hsa-miR-9, hsa-miR-767, hsa-miR-449a, hsa-miR-130a-3p, hsa-miR-214-3p, hsa-miR-452-5p, hsa-miR-224-5p, hsa-miR-483-5p, hsa-miR-483-3p, hsa-miR-9-3p and hsa-miR-767-5p.

21. The method of claim 15, wherein underexpression or nonexpression of one or more microRNAs selected from hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, and hsa-miR-99b-5p in the biological sample from the subject in comparison to a control sample from an individual known not to have POAG predicts a negative outcome or onset and/or progression and/or severity and/or recurrence of POAG.

22. The method of claim 21, further comprising administering to the subject an inhibitory nucleic acid that reduces or inhibits the expression of one or more microRNAs selected from hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, and hsa-miR-99b-5p.

23. The method of claim 21, further comprising administering to the subject one or more microRNAs or one or more mimics of microRNAs selected from hsa-miR-100, hsa-miR-100-5p, hsa-miR-105, hsa-miR-105-5p, hsa-miR-1226, hsa-miR-1226-3p, hsa-miR-124, hsa-miR-124-3p, hsa-miR-124-5p, hsa-miR-1250, hsa-miR-129, hsa-miR-129-5p, hsa-miR-138, hsa-miR-138-1, hsa-miR-138-2, hsa-miR-138-2-3p, hsa-miR-139, hsa-miR-139-5p, hsa-miR-181b, hsa-miR-181b-5p, hsa-miR-18a, hsa-miR-18a-3p, hsa-miR-18b, hsa-miR-18b-5p, hsa-miR-193b, hsa-miR-193b-5p, hsa-miR-19b, hsa-miR-19b-1, hsa-miR-19b-1-5p, hsa-miR-211, hsa-miR-211-5p, hsa-miR-219, hsa-miR-219-1, hsa-miR-219-2, hsa-miR-219-2-3p, hsa-miR-219-5p, hsa-miR-2276, hsa-miR-2277, hsa-miR-2277-3p, hsa-miR-30b, hsa-miR-30b-3p, hsa-miR-3117, hsa-miR-3117-3p, hsa-miR-3182, hsa-miR-323b, hsa-miR-323b-3p, hsa-miR-34b, hsa-miR-34b-3p, hsa-miR-3613, hsa-miR-3613-3p, hsa-miR-3622a, hsa-miR-3622a-5p, hsa-miR-376a, hsa-miR-376a-5p, hsa-miR-4423, hsa-miR-4423-5p, hsa-miR-4640, hsa-miR-4640-3p, hsa-miR-4677, hsa-miR-4677-3p, hsa-miR-505, hsa-miR-505-5p, hsa-miR-513c, hsa-miR-513c-5p, hsa-miR-545, hsa-miR-545-5p, hsa-miR-548ah, hsa-miR-548ah-3p, hsa-miR-548ah-5p, hsa-miR-99b, and hsa-miR-99b-5p.

24. The method of claim 15, wherein the individual is symptomatic for POAG.

25. The method of claim 15, wherein the individual has a family history of POAG.

26. The method of claim 15, wherein said output of the predictive model predicts a likelihood of onset and/or progression and/or severity and/or recurrence of POAG in the individual after said individual has undergone treatment for POAG.

27. The method of claim 15, further comprising providing a report having a prediction of onset and/or progression and/or severity and/or recurrence of POAG of said individual.

28. The method of claim 15, further comprising combining the allelic information and/or gene expression levels of said signature biomarkers with one or more other biomarkers to predict onset and/or progression and/or severity and/or recurrence of POAG in said individual.

29. The method of claim 15, wherein the expression levels of a collection of signature biomarkers comprise gene expression levels is measured at multiple times.

30. The method of claim 29, further comprising using the dynamics of the gene expression levels measured at multiple times to predict onset and/or progression and/or severity and/or recurrence of disease in said subject.

31. The method of claim 15, further comprising evaluating the output of the predictive model to determine whether or not the individual falls in a high risk group.

32. The method of claim 15, further comprising developing said predictive model using stability selection or logistic regression.

33. The method of claim 15, wherein applying said allelic information and/or expression levels of the collection of signature biomarkers to said predictive model comprises weighting said expression levels according to stability rankings or predictive power rankings of the collection of signature biomarkers.
